(19) United States
(12) Patent Application Publication
(10) Pub. No.: US 2021/0338681 A1
(43) Pub. Date: Nov. 4, 2021

(54) COMBINATION THERAPIES

(71) Applicant: SECURA BIO, INC., Las Vegas, NV (US)

(72) Inventors: Jonathan A. PACHTER, Wayland, MA (US); Shawn LELAND, Aurora, CO (US)

(21) Appl. No.: 17/282,058

(22) PCT Filed: Oct. 1, 2019
(86) PCT No.: PCT/US2019/053993

§ 371 Date: Apr. 1, 2021

Related U.S. Application Data
(60) Provisional application No. 62/739,658, filed on Oct. 1, 2018.

(51) Int. Cl. A61K 31/52; C07K 16/28; A61P 35/00

(52) U.S. Cl. CPC A61K 31/52; A61P 35/00; C07K 16/2803

(57) ABSTRACT

Provided herein are pharmaceutical compositions comprising a phosphatidylinositol 3-kinase inhibitor, or a pharmaceutically acceptable form thereof, in combination with a CD47 inhibitor, or a pharmaceutically acceptable form thereof in the presence or absence of an opsonizing antibody. Also provided herein are methods of treatment comprising administration of the compositions, and uses of the compositions, e.g., for treatment of cancer.

### CROSS REFERENCE TO RELATED APPLICATIONS

This application claims priority from U.S. Provisional Patent Application No. 62/739,658 filed on Oct. 1, 2018, which is incorporated herein by reference in its entirety.

### BACKGROUND

The phosphoinositide 3-kinases (PI3Ks) signaling pathway is one of the most highly mutated systems in human cancers. PI3Ks are members of a unique and conserved family of intracellular lipid kinases that phosphorylate the 3′-OH group on phosphatidylinositols or phosphoinositides. The PI3K family comprises 15 kinases with distinct substrate specificities, expression patterns, and modes of regulation. The class I PI3Ks (p110α, p110β, p10δ, and p110γ) are typically activated by tyrosine kinases or G-protein coupled receptors to generate phosphatidylinositol (3,4,5)-trisphosphate (PIP3), which engages downstream effectors such as those in the AKT/PDK1 pathway, mTOR, the Tec family kinases, and the Rho family GTPases. The class II and III PI3Ks play a key role in intracellular trafficking through the synthesis of phosphatidylinositol 3-bisphosphate (PI(3)P) and phosphatidylinositol (3,4)-bisphosphate (PI(3,4)P2). The PI3Ks are protein kinases that control cell growth (mTORC1) or monitor genomic integrity (ATM, ATR, DNA-PK, and hSmg-1).

There are four mammalian isoforms of class I PI3Ks: PI3K-α, β, δ (class Ia PI3Ks) and PI3K-γ (a class Ib PI3K). These enzymes catalyze the production of PIP3, leading to activation of downstream effector pathways important for cellular survival, differentiation, and function. PI3K-α and PI3K-β are widely expressed and are important mediators of signaling from cell surface receptors. PI3K-α is the isoform most often found mutated in cancers and has a role in insulin signaling and glucose homeostasis (Knight et al. Cell (2006) 125(4): 733-47; Vanhaesebroeck et al. Current Topic Microbiol. Immunol. (2010) 347: 1-19). PI3K-β is activated in cancers where phosphatase and tensin homolog (PTEN) is deleted. Both isoforms are targets of small molecule therapeutics in development for cancer.

PI3K-δ and -γ are preferentially expressed in leukocytes and are important in leukocyte function. These isoforms also contribute to the development and maintenance of hematologic malignancies (Vanhaesebroeck et al. Current Topic Microbiol. Immunol. (2010) 347: 1-19; Clayton et al. J. Exp. Med. (2002) 196(6): 753-63; Fung-Leung Cell Signal (2011) 23(4): 603-8; Okkenhaug et al. Science (2002) 297(5583): 1031-34). PI3K-δ is activated by cellular receptors (e.g., receptor tyrosine kinases) through interaction with the Sarc homology 2 (SH2) domains of the PI3K regulatory subunit (p85), or through direct interaction with RAS.

### SUMMARY

Provided herein, at least in part, are methods and compositions comprising a PI3K inhibitor in combination with a CD47 inhibitor. In some embodiments, the combinations of a PI3K inhibitor with a CD47 inhibitor can have a synergistic effect in treating a cancer (e.g., in reducing cancer cell growth or viability, or both). In some embodiments, the combination of a PI3K inhibitor and a CD47 inhibitor can allow the MK inhibitor and/or the CD47 inhibitor to be administered at a lower dosage than would be required to achieve the same therapeutic effect compared to a monotherapy dose. In some embodiments, the combinations can allow the PI3K inhibitor, the CD47 inhibitor, or both, to be administered at a lower frequency than if the PI3K inhibitor or the CD47 inhibitor were administered as a monotherapy. Such combinations can provide advantageous effects, e.g., reducing, preventing, delaying, and/or decreasing in the occurrence of one or more side effects, toxicity, or resistance that would otherwise be associated with administration of a higher dose of the inhibitors.

In an aspect, described herein is a method of treating, managing, or preventing cancer in a subject comprising administering to the subject a therapeutically effective amount of a PI3K inhibitor in combination with a CD47 inhibitor.

In some embodiments, the CD47 inhibitor reduces CD47/SIRPα interaction. In some embodiments, the CD47 inhibitor is a protein comprising a CD47 binding domain of SIRPα linked to an immunoglobulin Fc region. In some embodiments, the CD47 inhibitor is an anti-CD47 antibody. In some embodiments, the CD47 inhibitor is chosen from B6H12, Hu5F9-G4, TTI-621, CC-90002, TI-061, ALX-148, SRF-231, IF8 Ab, or 13H3 Ab, or a combination thereof. In some embodiments, the CD47 inhibitor is 13H3 Ab. In some embodiments, the CD47 inhibitor is B6H12.

In some embodiments, the PI3K inhibitor is a PI3K-alpha inhibitor, PI3K-beta inhibitor, PI3K-gamma inhibitor, PI3K-delta inhibitor, or PI3K-delta/gamma dual inhibitor. In some embodiments, the PI3K inhibitor is a PI3K-gamma inhibitor. In some embodiments, the PI3K inhibitor is a PI3K-delta/gamma dual inhibitor. In some embodiments, the PI3K inhibitor is chosen from tenalisib, duvelisib, idelalisib, copanlisib, IPI-549, CAL-130, BKM 120. GDC-0941, PX-866, GDC-0032, BAY 80-6946, BEZ235, BYL719, BGT-226, GDC-0980, GSK 2126458, PF-05212384, XL765, AS604850, AS252424, or XL147, or a combination thereof. In some embodiments, the PI3K inhibitor is tenalisib. In some embodiments, the PI3K inhibitor is IPI-549. In some embodiments, the PI3K inhibitor is duvelisib.

In some embodiments, the PI3K inhibitor and the CD47 inhibitor are the only therapeutically active ingredients. In some embodiments, the PI3K inhibitor and the CD47 inhibitor are in a single dosage form. In some embodiments, the PI3K inhibitor and the CD47 inhibitor are in separate dosage forms. In some embodiments, the PI3K inhibitor and the CD47 inhibitor is synergistic in treating a cancer. In some embodiments, the anti-cancer effect provided by a combination of the PI3K inhibitor and the CD47 inhibitor is greater than the anti-cancer effect relative to a monotherapy with the same dose of the PI3K inhibitor. In some embodiments, the anti-cancer effect provided by a combination of the PI3K inhibitor and the CD47 inhibitor is greater than the anti-cancer effect relative to a monotherapy with the same dose of the CD47 inhibitor.

In some embodiments, the PI3K inhibitor is administered concurrently with the CD47 inhibitor. In some embodiments, the PI3K inhibitor is administered subsequent to the CD47 inhibitor. In some embodiments, the PI3K inhibitor is administered prior to the CD47 inhibitor.

In an aspect, described herein is a method of improving the efficacy of a CD47 inhibitor, the method comprising administering to a subject having cancer a therapeutically effective amount of a PI3K inhibitor in combination with the CD47 inhibitor.

In some embodiments, the CD47 inhibitor reduces CD47/SIRPα interaction. In some embodiments, the CD47 inhibitor is a protein comprising a CD47 binding domain of SIRPα linked to an immunoglobulin Fc region. In some embodiments, the CD47 inhibitor is an anti-CD47 antibody. In some embodiments, the CD47 inhibitor is chosen from B6H12, Hu5F9-G4, TTI-621, CC-90002, TI-061, ALX-148, SRF-231, IF8 Ab, or 13H3 Ab, or a combination thereof. In some embodiments, the CD47 inhibitor is 13H3 Ab. In some embodiments, the CD47 inhibitor is B6H12.

In some embodiments, the PI3K inhibitor is a PI3K-alpha inhibitor, PI3K-beta inhibitor, PI3K-gamma inhibitor, PI3K-delta inhibitor, or PI3K-delta/gamma dual inhibitor. In some embodiments, the PI3K inhibitor is a PI3K-gamma inhibitor. In some embodiments, the PI3K inhibitor is a PI3K-delta/gamma dual inhibitor. In some embodiments, the PI3K inhibitor is chosen from tenalisib, duvelisib, idelalisib, copanlisib, IPI-549, CAL-130, BKM 120. GDC-0941, PX-866, GDC-0032, BAY 80-6946, BEZ235, BYL719, BGT-226, GDC-0980, GSK 2126458, PF-05212384, XL765, AS604850, AS252424, or XL147, or a combination thereof. In some embodiments, the PI3K inhibitor is tenalisib. In some embodiments, the PI3K inhibitor is IPI-549. In some embodiments, the PI3K inhibitor is duvelisib.

In an aspect, described herein is a method of improving the efficacy of a CD47 inhibitor, the method comprising:
administering to a subject having cancer a therapeutically effective amount of a CD47 inhibitor for a first period of time;
after the first period of time, administering to the subject having cancer a therapeutically effective amount of a combination therapy comprising the PI3K inhibitor in combination with a CD47 inhibitor for a second period of time; and
optionally repeating steps (a) and (b) one or more times.

In some embodiments, the CD47 inhibitor reduces CD47/SIRPα interaction. In some embodiments, the CD47 inhibitor is a protein comprising a CD47 binding domain of SIRPα linked to an immunoglobulin Fc region. In some embodiments, the CD47 inhibitor is an anti-CD47 antibody. In some embodiments, the CD47 inhibitor is chosen from B6H12, Hu5F9-G4, TTI-621, CC-90002, TI-061, ALX-148, SRF-231, IF8 Ab, or 13H3 Ab, or a combination thereof. In some embodiments, the CD47 inhibitor is 13H3 Ab. In some embodiments, the CD47 inhibitor is B6H12.

In some embodiments, the PI3K inhibitor is a PI3K-alpha inhibitor, PI3K-beta inhibitor, PI3K-gamma inhibitor, PI3K-delta inhibitor, or PI3K-delta/gamma dual inhibitor. In some embodiments, the PI3K inhibitor is a PI3K-gamma inhibitor. In some embodiments, the PI3K inhibitor is a PI3K-delta/gamma dual inhibitor. In some embodiments, the PI3K inhibitor is chosen from tenalisib, duvelisib, idelalisib, copanlisib, IPI-549, CAL-130, BK1\4 120, GDC-0941, PX-866, GDC-0032, BAY 80-6946, BEZ235, BYL719, BGT-226. (31)(7-0980, GSK 2126458, PF-05212384, XI-765, AS604850, AS252424, or XL147, or a combination thereof. In some embodiments, the PI3K inhibitor is tenalisib. In some embodiments, the PI3K inhibitor is IPI-549. In some embodiments, the PI3K inhibitor is duvelisib.

In an aspect, described herein is a method of reducing a hematologic-related side effect and/or toxicity in a subject having cancer and receiving treatment with a CD47 inhibitor for the cancer, the method comprising administering to the subject a therapeutically effective amount of a PI3K inhibitor in combination with the CD47 inhibitor.

In some embodiments, the CD47 inhibitor reduces CD47/SIRPα interaction. In some embodiments, the CD47 inhibitor is a protein comprising a CD47 binding domain of SIRPα linked to an immunoglobulin Fc region. In some embodiments, the CD47 inhibitor is an anti-CD47 antibody. In some embodiments, the CD47 inhibitor is chosen from B6H12, Hu5F9-G4, TTI-621, CC-90002, TI-061, ALX-148, SRF-231, IF8 Ab, or 13H3 Ab, or a combination thereof. In some embodiments, the CD47 inhibitor is 13H3 Ab. In some embodiments, the CD47 inhibitor is B6H12.

In some embodiments, the PI3K inhibitor is a PI3K-alpha inhibitor, PI3K-beta inhibitor, PI3K-gamma inhibitor, PI3K-delta inhibitor, or PI3K-delta/gamma dual inhibitor. In some embodiments, the PI3K inhibitor is a PI3K-gamma inhibitor. In some embodiments, the PI3K inhibitor is a PI3K-delta/gamma dual inhibitor. In some embodiments, the PI3K inhibitor is chosen from tenalisib, duvelisib, idelalisib, copanlisib, IPI-549, CAL-I30, BKM 120, GDC-0941, PX-866, GDC-0032, BAY 80-6946, BEZ235, BYL719, BGT-226, GDC-0980, GSK 2126458, PF-05212384, XL765, AS604850, AS252424. or XL147, or a combination thereof. In some embodiments, the PI3K inhibitor is tenalisib. In some embodiments, the PI3K inhibitor is IPI-549. In some embodiments, the PI3K inhibitor is duvelisib.

In an aspect, described herein is a method of delaying or decreasing resistance of a subject having cancer, the method comprising administering to the subject a therapeutically effective amount of a PI3K inhibitor in combination with a CD47 inhibitor, wherein the resistance is resistance to a reduction of CD47/SIRPα interaction.

In some embodiments, the CD47 inhibitor reduces CD47/SIRPα interaction. In some embodiments, the CD47 inhibitor is a protein comprising a CD47 binding domain of SIRPα linked to an immunoglobulin Fc region. In some embodiments, the CD47 inhibitor is an anti-CD47 antibody. In some embodiments, the CD47 inhibitor is chosen from B6H12, Hu5F9-G4, TTI-621, CC-90002, TI-061, ALX-148, SRF-231, IF8 Ab, or 13H3 Ab, or a combination thereof. In some embodiments, the CD47 inhibitor is 13H3 Ab. In some embodiments, the CD47 inhibitor is B6H12.

In some embodiments, the PI3K inhibitor is a PI3K-alpha inhibitor, PI3K-beta inhibitor, PI3K-gamma inhibitor, PI3K-delta inhibitor, or PI3K-delta/gamma dual inhibitor. In some embodiments, the PI3K inhibitor is a PI3K-gamma inhibitor. In some embodiments, the PI3K inhibitor is a PI3K-delta/gamma dual inhibitor. In some embodiments, the PI3K inhibitor is chosen from tenalisib, duvelisib, idelalisib, copanlisib, IPI-549, CAL-130, BKM 120. GDC-0941, PX-866, GDC-0032, BAY 80-6946, BEZ235, BYL719, BGT-226, GDC-0980, GSK 2126458, PF-05212384, XL765, AS604850, AS252424, or XL147, or a combination thereof. In some embodiments, the PI3K inhibitor is tenalisib. In some embodiments, the PI3K inhibitor is IPI-549. In some embodiments, the PI3K inhibitor is duvelisib.

In some embodiments, the cancer is of hematopoietic origin.

In some embodiments, the cancer is lymphoma or leukemia. In some embodiments, the cancer is B-cell lymphoma, mantle cell lymphoma, non-Hodgkin's lymphoma, non-Hodgkin's B-cell lymphoma, or T-cell lymphoma. In some embodiments, the cancer is multiple myeloma. In some embodiments, the cancer is non-Hodgkin's lymphoma. In some embodiments, the non-Hodgkin's lymphoma is B-cell non-Hodgkin's lymphoma. In some embodiments, the B-cell non-Hodgkin's lymphoma is diffuse large B-cell lymphoma. In some embodiments, the diffuse large B-cell lymphoma is diffuse large B-cell lymphoma activated B-cell-like or diffuse large B-cell lymphoma germinal center B-cell-like. In some embodiments, the B-cell non-Hodgkin's lymphoma is marginal zone lymphoma. In some embodiments, the cancer is indolent non-Hodgkin's lymphoma. In some embodiments, the indolent non-Hodgkin's lymphoma is chronic lymphocytic leukemia/small lymphocytic lymphoma. In some embodiments, the cancer is follicular lymphoma. In some embodiments, the cancer is acute myeloid leukemia. In some embodiments, the cancer is T-cell lymphoma. In some embodiments, the T-cell lymphoma is peripheral T-cell lymphoma.

In some embodiments, the cancer is a solid tumor. In some embodiments, the solid tumor is an ovarian cancer, pancreatic cancer, breast cancer, prostate cancer, colorectal cancer, non-small cell lung cancer, squamous cell carcinoma, or hepatocellular carcinoma.

In some embodiments, the subject is a human.

In some embodiments, the methods as described herein further comprises administering an opsonizing antibody to the subject. In some embodiments, the opsonizing antibody is an anti-CD20 antibody (e.g., rituximab, obinutuzumab, ofatumumab), an anti-EGFR antibody (e.g., cetuximab, panitumumab), an anti-HER2 antibody (e.g., trastuzumab, pertuzumab), an anti-CD38 antibody (e.g., daratumumab, isatuximab), an anti-CD19 antibody (e.g., tafasitamab), an anti-CD22 antibody (e.g., moxetumomab pasudotox), an anti-CCR4 antibody (e.g., mogamulizumab), or any other opsonizing antibody.

In an aspect, described herein is a composition comprising a combination of a PI3K inhibitor and a CD47 inhibitor.

In some embodiments, the CD47 inhibitor reduces CD47/SIRPα interaction. In some embodiments, the CD47 inhibitor is a protein comprising a CD47 binding domain of SIRPα linked to an immunoglobulin Fc region. In some embodiments, the CD47 inhibitor is an anti-CD47 antibody. In some embodiments, the CD47 inhibitor is chosen from B6H12, Hu5F9-G4, TTI-621, CC-90002, TI-061, ALX-148, SRF-231, IF8 Ab, or 13H3 Ab, or a combination thereof. In some embodiments, the CD47 inhibitor is 13H3 Ab. In some embodiments, the CD47 inhibitor is B6H12.

In some embodiments, the PI3K inhibitor is a PI3K-alpha inhibitor, PI3K-beta inhibitor, PI3K-gamma inhibitor, PI3K-delta inhibitor, or PI3K-delta/gamma dual inhibitor. In some embodiments, the PI3K inhibitor is a PI3K-gamma inhibitor. In some embodiments, the PI3K inhibitor is a PI3K-delta/gamma dual inhibitor. In some embodiments, the PI3K inhibitor is chosen from tenalisib, duvelisib, idelalisib, copanlisib, IPI-549, CAL-I30, BKM 120, GDC-0941, PX-866, GDC-0032, BAY 80-6946, BEZ235, BYL719, BGT-226, GDC-0980, GSK 2126458, PF-05212384, XL765, AS604850, AS252424. or XL147, or a combination thereof. In some embodiments, the PI3K inhibitor is tenalisib. In some embodiments, the PI3K inhibitor is IPI-549. In some embodiments, the PI3K inhibitor is duvelisib.

### BRIEF DESCRIPTION OF THE DRAWINGS

FIG. 1 shows a graphical summary of duvelisib increasing phagocytosis by human macrophages as a single agent, and in combination with anti-CD47 (clone B6H12) and an opsonizing antibody.

FIG. 2 shows a graphical summary of duvelisib increasing phagocytosis by human macrophages in combination with anti-CD47 (clone 5F9).

### DETAILED DESCRIPT

### Combination of PI3K Inhibitors and CD47 Inhibitors

Provided herein, at least in part, are methods of treating, managing, or preventing a cancer in a subject, the methods comprising administering to the subject a therapeutically effective amount of a PI3K inhibitor, or a pharmaceutically acceptable form thereof, in combination with a CD47 inhibitor, or a pharmaceutically acceptable form thereof. Pharmaceutical compositions comprising a therapeutically effective amount of a PI3K inhibitor, or a pharmaceutically acceptable form thereof, and a CD47 inhibitor, or a pharmaceutically acceptable form thereof are also described herein.

In some embodiments, provided herein is a method of treating, managing, or preventing a cancer in a subject comprising administering to the subject a therapeutically effective amount of a PI3K inhibitor, or a pharmaceutically acceptable form thereof, in combination with a CD47 inhibitor, or a pharmaceutically acceptable form thereof, wherein the PI3K inhibitor is a PI3K gamma inhibitor.

some embodiments, provided herein is a pharmaceutical composition comprising a therapeutically effective amount of a PI3K inhibitor, or a pharmaceutically acceptable form thereof. and a CD47 inhibitor, or a pharmaceutically acceptable form thereof, wherein the PI3K inhibitor is a PI3K gamma inhibitor.

In some embodiments of the compositions and methods provided herein, the PI3K inhibitor is a PI3K gamma inhibitor, and the CD47 inhibitor is an anti-CD47 antibody or a protein comprising a CD47 binding domain of SIRPα linked to an immunoglobulin Fc region, or a combination thereof. In some embodiments, the anti-CD47 antibody is B6H12, Hu5F9-G4, CC-90002, TI-061, SRF-231, 1F8 Ab, or 13H3 Ab, or a combination thereof. In some embodiments, the protein comprising a CD47 binding domain of SIRPα linked to an immunoglobulin Fc region is TTI-621 or ALX-148, or a combination thereof.

In some embodiments, the PI3K inhibitor is a PI3K gamma inhibitor, and the CD47 inhibitor is B6H12. In some embodiments, the PI3K inhibitor is a PI3K gamma inhibitor, and the CD47 inhibitor is Hu5F9-G4. In some embodiments, the PI3K inhibitor is a PI3K gamma inhibitor, and the CD47 inhibitor is CC-90002. In some embodiments, the PI3K inhibitor is a PI3K gamma inhibitor, and the CD47 inhibitor is TI-061. In some embodiments, the PI3K inhibitor is a PI3K gamma inhibitor, and the CD47 inhibitor is ALX-148. In some embodiments, the PI3K inhibitor is a PI3K gamma inhibitor, and the CD47 inhibitor is SRF-231. In some embodiments, the PI3K inhibitor is a PI3K gamma inhibitor, and the CD47 inhibitor is 1F8 Ab. In some embodiments, the PI3K inhibitor is a PI3K gamma inhibitor, and the CD47 inhibitor is 13H3 Ab. In some embodiments, the PI3K inhibitor is a PI3K gamma inhibitor, and the CD47 inhibitor is TTI-621.

In some embodiments, provided herein is a method of treating, managing, or preventing a cancer in a subject comprising administering to the subject a therapeutically effective amount of a PI3K inhibitor, or a pharmaceutically acceptable form thereof, in combination with a CD47 inhibitor, or a pharmaceutically acceptable form thereof, wherein the PI3K inhibitor is IPI-549.

In some embodiments, provided herein is a pharmaceutical composition comprising a therapeutically effective amount of a PI3K inhibitor, or a pharmaceutically acceptable form thereof, and a CD47 inhibitor, or a pharmaceutically acceptable form thereof, wherein the PI3K inhibitor is IPI-549.

In some embodiments of the compositions and methods provided herein, the PI3K inhibitor is IPI-549, and the CD47 inhibitor is an anti-CD47 antibody or a protein comprising a CD47 binding domain of SIRPα linked to an immunoglobulin Fc region, or a combination thereof. In some embodiments, the anti-CD47 antibody is B6H12, Hu5F9-G4, CC-90002, TI-061, SRF-231, 1F8 Ab, or 13H3 Ab, or a combination thereof. In some embodiments, the protein comprising a CD47 binding domain of SIRPα linked to an immunoglobulin Fc region is TTI-621 or ALX-148.

In some embodiments, the PI3K inhibitor is IPI-549, and the CD47 inhibitor is B6H12. In some embodiments, the PI3K inhibitor is IPI-549, and the CD47 inhibitor is Hu5F9-G4. In some embodiments, the PI3K inhibitor is IPI-549, and the CD47 inhibitor is CC-90002. In some embodiments, the PI3K inhibitor is IPI-549, and the CD47 inhibitor is TI-061.

In some embodiments, the PI3K inhibitor is IPI-549, and the CD47 inhibitor is ALX-148. In some embodiments, the PI3K inhibitor is IPI-549, and the CD47 inhibitor is SRF-231. In some embodiments, the PI3K inhibitor is IPI-549, and the CD47 inhibitor is 1F8 Ab. In some embodiments, the PI3K inhibitor is IPI-549, and the CD47 inhibitor is 13H3 Ab. In some embodiments, the PI3K inhibitor is IPI-549, and the CD47 inhibitor is TTI-621.

In some embodiments, provided herein is a method of treating, managing, or preventing a cancer in a subject comprising administering to the subject a therapeutically effective amount of a PI3K inhibitor, or a pharmaceutically acceptable form thereof, in combination with a CD47 inhibitor, or a pharmaceutically acceptable form thereof, wherein the PI3K inhibitor is a PI3K delta/gamma dual inhibitor.

In some embodiments, provided herein is a pharmaceutical composition comprising a therapeutically effective amount of a PI3K inhibitor, or a pharmaceutically acceptable form thereof, and a CD47 inhibitor, or a pharmaceutically acceptable form thereof, wherein the PI3K inhibitor is a PI3K delta/gamma dual inhibitor.

In some embodiments of the compositions and methods provided herein, the PI3K inhibitor is a PI3K delta/gamma dual inhibitor, and the CD47 inhibitor is an anti-CD47 antibody or a protein comprising a CD47 binding domain of SIRPα linked to an immunoglobulin Fc region, or a combination thereof. In some embodiments, the anti-CD47 antibody is B6H12, Hu5F9-G4, CC-90002, TI-061, SRF-231, 1F8 Ab, or 13H3 Ab, or a combination thereof. In some embodiments, the protein comprising a CD47 binding domain of SIRPα linked to an immunoglobulin Fc region is TTI-621 or ALX-148.

In some embodiments, the PI3K inhibitor is a PI3K delta/gamma dual inhibitor, and the CD47 inhibitor is B6H12. In some embodiments, the PI3K inhibitor is a PI3K delta/gamma dual inhibitor, and the CD47 inhibitor is Hu5F9-G4. In some embodiments, the PI3K inhibitor is a PI3K delta/gamma dual inhibitor, and the CD47 inhibitor is CC-90002. In some embodiments, the PI3K inhibitor is a PI3K delta/gamma dual inhibitor, and the CD47 inhibitor is TI-061. In some embodiments, the PI3K inhibitor is a PI3K delta/gamma dual inhibitor, and the CD47 inhibitor is ALX-148. In some embodiments, the PI3K inhibitor is a PI3K delta/gamma dual inhibitor, and the CD47 inhibitor is SRF-231. In some embodiments, the PI3K inhibitor is a PI3K delta/gamma dual inhibitor, and the CD47 inhibitor is 1F8 Ab. In some embodiments, the PI3K inhibitor is a PI3K delta/gamma dual inhibitor, and the CD47 inhibitor is 13H3 Ab. In some embodiments, the PI3K inhibitor is a PI3K delta/gamma dual inhibitor, and the CD47 inhibitor is TTI-621.

In some embodiments, provided herein is a method of treating, managing, or preventing a cancer in a subject comprising administering to the subject a therapeutically effective amount of a PI3K inhibitor, or a pharmaceutically acceptable form thereof, in combination with a CD47 inhibitor, or a pharmaceutically acceptable form thereof, wherein the MK inhibitor is tenalisib.

In some embodiments, provided herein is a pharmaceutical composition comprising a therapeutically effective amount of a PI3K inhibitor, or a pharmaceutically acceptable form thereof, and a CD47 inhibitor, or a pharmaceutically acceptable form thereof, wherein the PI3K inhibitor is tenalisib.

In some embodiments of the compositions and methods provided herein, the PI3K inhibitor is tenalisib, and the CD47 inhibitor is an anti-CD47 antibody or a protein comprising a CD47 binding domain of SIRPα linked to an immunoglobulin Fc region, or a combination thereof. In some embodiments, the anti-CD47 antibody is B6H12, Hu5F9-G4, CC-90002, TI-061, SRF-231, 1F8 Ab, or 13H3 Ab, or a combination thereof. In some embodiments, the protein comprising a CD47 binding domain of SIRPα linked to an immunoglobulin Fc region is TTI-621 or ALX-148.

In some embodiments, the PI3K inhibitor is tenalisib, and the CD47 inhibitor is B6H12. In some embodiments, the PI3K inhibitor is tenalisib, and the CD47 inhibitor is Hu5F9-G4. In some embodiments, the PI3K inhibitor is tenalisib, and the CD47 inhibitor is CC-90002. In some embodiments, the PI3K inhibitor is tenalisib, and the CD47 inhibitor is TI-061. In some embodiments, the PI3K inhibitor is tenalisib, and the CD47 inhibitor is ALX-148.

In some embodiments, the PI3K inhibitor is tenalisib, and the CD47 inhibitor is SRF-231. In some embodiments, the PI3K inhibitor is tenalisib, and the CD47 inhibitor is 1F8 Ab. In some embodiments, the PI3K inhibitor is tenalisib, and the CD47 inhibitor is 13H3 Ab. In some embodiments, the PI3K inhibitor is tenalisib, and the CD47 inhibitor is TTI-621.

In some embodiments, provided herein is a method of treating, managing, or preventing a cancer in a subject comprising administering to the subject a therapeutically effective amount of a PI3K inhibitor, or a pharmaceutically acceptable form thereof, in combination with a CD47 inhibitor, or a pharmaceutically acceptable form thereof, wherein the PI3K inhibitor is duvelisib.

In some embodiments, provided herein is a pharmaceutical composition comprising a therapeutically effective amount of a PI3K inhibitor, or a pharmaceutically acceptable form thereof, and a CD47 inhibitor, or a pharmaceutically acceptable form thereof, wherein the PI3K inhibitor is duvelisib.

In some embodiments of the compositions and methods provided herein, the PI3K inhibitor is duvelisib, and the CD47 inhibitor is an anti-CD47 antibody or a protein comprising a CD47 binding domain of SIRPα linked to an immunoglobulin Fc region, or a combination thereof. In some embodiments, the anti-CD47 antibody is B6H12, Hu5F9-G4, CC-90002, TI-061, SRF-231, 1F8 Ab, or 13H3 Ab, or a combination thereof. In some embodiments, the protein comprising a CD47 binding domain of SIRPα linked to an immunoglobulin Fc region is TTI-621or ALX-148.

In some embodiments, the PI3K inhibitor is duvelisib, and the CD47 inhibitor is B6H12. In some embodiments, the PI3K inhibitor is duvelisib, and the CD47 inhibitor is Hu5F9-G4. In some embodiments, the PI3K inhibitor is duvelisib, and the CD47 inhibitor is CC-90002. In some embodiments, the PI3K inhibitor is duvelisib, and the CD47 inhibitor is TI-061. In some embodiments, the PI3K inhibitor is duvelisib, and the CD47 inhibitor is ALX-148. In some embodiments, the PI3K inhibitor is duvelisib, and the CD47 inhibitor is SRF-231. In some embodiments, the PI3K inhibitor is duvelisib, and the CD47 inhibitor is 1F8 Ab. In some embodiments, the PI3K inhibitor is duvelisib, and the CD47 inhibitor is 13H3 Ab. In some embodiments, the PI3K inhibitor is duvelisib, and the CD47 inhibitor is TTI-621.

In some embodiments, the CD47 inhibitor, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g. , duvelisib, tenalisib, IPI549), or a pharmaceutically acceptable form thereof, is administered. In another embodiment, the CD47 inhibitor, or a pharmaceutically acceptable form thereof, is administered concurrently with the PI3K inhibitor (e.g., duvelisib, tenalisib, IPI-549), or a pharmaceutically acceptable form thereof, in a single dosage form or separate dosage forms. In another embodiment, the CD47 inhibitor, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., duvelisib, tenalisib, IPI-549), or a pharmaceutically acceptable form thereof, is administered.

In certain embodiments, the P13K inhibitor (e.g. duvelisib, tenalisib, 549), or a pharmaceutically acceptable form thereof, and the CD47 inhibitor are administered via a same route, e.g., both are administered orally. In other embodiments, the PI3K inhibitor (e.g. , Compound ,1 tenalisib, IPI-549), or a pharmaceutically acceptable form thereof, and the CD47 inhibitor are administered via different routes, e.g., one is administered orally and the other is administered intravenously.

In certain embodiments, the PI3K inhibitor (e.g. , duvelisib, tenalisib, 549), or a pharmaceutically acceptable form thereof, and the CD47 inhibitor, or a pharmaceutically acceptable form thereof, are the only therapeutically active ingredients of the compositions and methods provided herein. In some embodiments, the PI3K inhibitor (e.g. , duvelisib, tenalisib, IPI-549), or a pharmaceutically acceptable form thereof, and the CD47 inhibitor, or a pharmaceutically acceptable form thereof, are further administered with an opsonizing antibody. In some embodiments, the opsonizing antibody is an anti-CD20 antibody (e.g., rituximab, obinutuzumab, ofatumumab), an anti-EGFR antibody (e.g., cetuximab, panitumumab), an anti-HER2 antibody (e.g., trastuzumab, pertuzumab), an anti-CD38 antibody (e.g., daratumumab, isatuximab), an anti-CD19 antibody (e.g., tafasitamab), an anti-CD22 antibody (e.g., moxetumomab pasudotox), an anti-CCR4 antibody (e.g., mogamulizumab), or any other opsonizing antibody.

### Cancers

The diseases or disorders (e.g., cancer) that can be treated, managed, or prevented with a pharmaceutical composition as provided herein, or according to the methods as provided herein, include, but are not limited to, breast cancer such as a ductal carcinoma, lobular carcinoma, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epithelioid carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, NK cell leukemia (e.g., blastic plasmacytoid dendritic cell neoplasm), acute myelogenous leukemia, (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer; kidney cancer; thyroid cancer such as papillary, follicular, medullary and anaplastic; lymphoma such as diffuse large B-cell lymphoma, B-cell immunoblastic lymphoma, NK cell lymphoma (e.g., blastic plasmacytoid dendritic cell neoplasm), and Burkitt lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system cancers (CNS) such as primary brain tumor, which includes gliomas (astrocytoma, anaplastic astrocytoma, or glioblastoma multiforme), oligodendroglioma, ependymoma, meningioma, lymphoma, schwannoma, and medulloblastoma; peripheral nervous system (PNS) cancers such as acoustic neuromas and malignant peripheral nerve sheath tumor (MPNST) including neurofibromas and schwannomas, malignant fibrocytoma, malignant fibrous histiocytoma, malignant meningioma, malignant mesothelioma, and malignant mixed Miillerian tumor; oral cavity and oropharyngeal cancers such as, hypopharyngeal cancer, laryngeal cancer, nasopharyngeal cancer, and oropharyngeal cancer; stomach cancers such as lymphomas, gastric stromal tumors, and carcinoid tumors; testicular cancers such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin lymphoma, non-Hodgkin lymphomas carcinoids or carcinoid tumors; rectal cancer; and colon cancer.

In some embodiments, the cancer or disease is a blood disorder or a hematologic malignancy.

In some embodiments, the cancer or disease is selected from one or more of the following: acoustic neuroma, adenocarcinoma, adrenal gland cancer, anal cancer, angiosarcoma (e.g., lymphangiosarcoma, lymphangioendotheliosarcoma, hemangiosarcoma), benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer, esophageal cancer (e.g., adenocarcinoma of the esophagus, Barrett's adenocarinoma), Ewing sarcoma, familiar hypereosinophilia, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphocytic leukemia (ALL), which includes B-lineage ALL and T-lineage ALL, chronic lymphocytic leukemia (CLL), prolymphocyte leukemia (PLL), hairy cell leukemia (HCL) and Waldenstrom's macroglobulinemia (WM); peripheral T cell lymphomas (PTCL), adult T cell leukemia/lymphoma (ATL), cutaneous T-cell lymphoma (CTCL), large granular lymphocytic leukemia (LGF), Hodgkin's disease and Reed-Stemberg disease; acute myelocytic leukemia (AML), chronic myelocytic leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), leiomyosarcoma (LMS), mastocytosis (e.g., systemic mastocytosis), multiple myeloma (MM), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), Paget's disease of the vulva, Paget's disease of the penis, papillary ad.enocarcinoma, pancreatic cancer (e.g., pancreatic andenocarcinoma, intraductal papillary mucinous neoplasm (IPMN)), pinealoma, primitive neuroectodermal tumor (PNT), prostate cancer (e.g., prostate adenocarcinoma), rhabdomyosarcoma, retinoblastoma, salivary gland, cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), and Waldenstrom's macroglobulinemia.

In some embodiments, the cancer or disease is a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others.

In some embodiments, the blood disorder or the hematologic malignancy includes, but is not limited to, acute lymphoblastic; leukemia ALL), T-cell ALL (T-ALL), B-cell ALL (B-ALL), acute myeloid leukemia (AML), chronic lymphocytic leukemia (CLL), chronic myelogenous leukemia (CML), blast phase CML, small lymphocytic lymphoma (SLL), CLL/SLL, blast phase CLL, Hodgkin lymphoma (HL), non-Hodgkin lymphoma (NHL), B-cell NHL, T-cell NHL, indolent NHL (iNHL), diffuse large B-cell lymphoma (DLBCL), mantle cell lymphoma (MCL), aggressive B-cell NHL, B-cell lymphoma (BCL), Richter's syndrome (RS), T-cell lymphoma (TCL), peripheral T-cell lymphoma (PTCL), cutaneous T-cell lymphoma (CTCL), transformed mycosis fungoides, Sezary syndrome, anaplastic large-cell lymphoma (ALCL), follicular lymphoma (FL), Waldenstrom macroglobulinemia (WM), lymphoplasmacytic lymphoma, Burkitt lymphoma, multiple myeloma (MM), amyloidosis, MPD, essential thrombocytosis (ET), myelofibrosis (MF), polycythemia vera (PV), chronic myelomonocytic leukemia (CMML), myelodysplastic syndrome (MDS), angioimmunoblastic lymphoma, high-risk MDS, and low-risk MDS. In some embodiments, the hematologic malignancy is relapsed. In some embodiments, the hematologic malignancy is refractory. In some embodiments, the cancer or disease is in a pediatric patient (including an infantile patient). In some embodiments, the cancer or disease is in an adult patient. Additional embodiments of a cancer or disease being treated or prevented by methods, compositions, or kits provided herein are described herein elsewhere.

In exemplary embodiments, the cancer or hematologic malignancy is CLL. In exemplary embodiments, the cancer or hematologic malignancy is CLL/SLL. In exemplary embodiments, the cancer or hematologic malignancy is blast phase CLL. In exemplary embodiments, the cancer or hematologic malignancy is SLL.

In exemplary embodiments, the cancer or hematologic malignancy is iNHL. In exemplary embodiments, the cancer or hematologic malignancy is DLBCL. In exemplary embodiments, the cancer or hematologic malignancy is B-cell NHL (e.g., aggressive B-cell NHL). In exemplary embodiments, the cancer or hematologic malignancy is MCL. In exemplary embodiments, the cancer or hematologic malignancy is RS. In exemplary embodiments, the cancer or hematologic malignancy is AML. In exemplary embodiments, the cancer or hematologic malignancy is MM. In exemplary embodiments, the cancer or hematologic malignancy is ALL. In exemplary embodiments, the cancer or hematologic malignancy is T-ALL. In exemplary embodiments, the cancer or hematologic malignancy is B-ALL. In exemplary embodiments, the cancer or hematologic malignancy is TCL. In exemplary embodiments, the cancer or hematologic malignancy is ALCL. In exemplary embodiments, the cancer or hematologic malignancy is leukemia. In exemplary embodiments, the cancer or hematologic malignancy is lymphoma. In exemplary embodiments, the cancer or hematologic malignancy is T-cell lymphoma. In exemplary embodiments, the cancer or hematologic malignancy is MDS (e.g., low grade MDS). In exemplary embodiments, the cancer or hematologic malignancy is MPD. In exemplary embodiments, the cancer or hematologic malignancy is a mast cell disorder. In exemplary embodiments, the cancer or hematologic malignancy is Hodgkin lymphoma (HL). In exemplary embodiments, the cancer or hematologic malignancy is non-Hodgkin lymphoma. In exemplary embodiments, the cancer or hematologic malignancy is PTCL. In exemplary embodiments, the cancer or hematologic malignancy is CTCL (e.g., mycosis fungoides or Sezary syndrome). In exemplary embodiments, the cancer or hematologic malignancy is WM. In exemplary embodiments, the cancer or hematologic malignancy is CML. In exemplary embodiments, the cancer or hematologic malignancy is FL. In exemplary embodiments, the cancer or hematologic malignancy is transformed mycosis fungoides. In exemplary embodiments, the cancer or hematologic malignancy is Sezary syndrome. In exemplary embodiments, the cancer or hematologic malignancy is acute T-cell leukemia. In exemplary embodiments, the cancer or hematologic malignancy is acute B-cell leukemia. In exemplary embodiments, the cancer or hematologic malignancy is Burkitt lymphoma. In exemplary embodiments, the cancer or hematologic malignancy is myeloproliferative neoplasms. In exemplary embodiments, the cancer or hematologic malignancy is splenic marginal zone. In exemplary embodiments, the cancer or hematologic malignancy is nodal marginal zone. In exemplary embodiments, the cancer or hematologic malignancy is extranodal marginal zone.

In some embodiments, the cancer or hematologic malignancy is a B cell lymphoma. In a specific embodiment, provided herein is a method of treating or managing a B cell lymphoma comprising administering to a patient a therapeutically effective amount of a compound provided herein, or a pharmaceutically acceptable derivative (e.g., salt or solvate) thereof. Also provided herein is a method of treating or lessening one or more of the symptoms associated with a B cell lymphoma comprising administering to a patient a therapeutically effective amount of a compound provided herein, or a pharmaceutically acceptable derivative (e.g., salt or solvate) thereof. In some embodiments, the B cell lymphoma is iNHL. In another embodiment, the B cell lymphoma is follicular lymphoma. In another embodiment, the B cell lymphoma, is Waldenstrom macroglobulinemia (lymphoplasmacytic lymphoma). In another embodiment, the B cell lymphoma is marginal zone lymphoma (MZL). In another embodiment, the B cell lymphoma is MCL. In another embodiment, the B cell lymphoma is HL. In another embodiment, the B cell lymphoma is aNHL. In another embodiment, the B cell lymphoma is DLBCL. In another embodiment, the B cell lymphoma is Richters lymphoma.

In some embodiments, the cancer or hematologic malignancy is a T cell lymphoma. In a specific embodiment, provided herein is a method of treating or managing a T cell lymphoma comprising administering to a patient a therapeutically effective amount of a compound provided herein, or a pharmaceutically acceptable derivative (e.g., salt or solvate) thereof. Also provided herein is a method of treating or lessening one or more of the symptoms associated with a T cell lymphoma comprising administering to a patient a therapeutically effective amount of a compound provided herein, or a pharmaceutically acceptable derivative (e.g., salt or solvate) thereof. In some embodiments, the T cell lymphoma is peripheral T cell lymphoma (PTCL). In another embodiment, the T cell lymphoma is cutaneous T cell lymphoma (CTCL).

In some embodiments, the cancer or hematologic malignancy is Sezary syndrome. In a specific embodiment, provided herein is a method of treating or managing Sezary syndrome comprising administering to a patient a therapeutically effective amount of a compound provided herein, or a pharmaceutically acceptable derivative (e.g., salt or solvate) thereof. Also provided herein is a method of treating or lessening one or more of the symptoms associated with Sezary syndrome comprising administering to a patient a therapeutically effective amount of a compound provided herein, or a pharmaceutically acceptable derivative (e.g., salt or solvate) thereof. The symptoms associated with Sezary syndrome include, but are not limited to, epidermotropism by neoplastic CD4+ lymphocytes, Pautrier's microabscesses, erythroderma, lymphadenopathy, atypical T cells in the peripheral blood, and hepatosplenomegaly.

The effectiveness of treatment in the preceding methods can for example be determined by measuring the decrease in size of tumors present in the patients with the neoplastic condition, or by assaying a molecular determinant of the degree of proliferation of the tumor cells.

Suitable test agents which can be tested in the preceding method include combinatorial libraries, defined chemical entities, peptide and peptide mimetics, oligonucleotides and natural product libraries, such as display (e.g., phage display libraries) and antibody products. Test agents may be used in an initial screen of, for example, 10 substances per reaction, and the substances of these batches which show inhibition or activation tested individually. Test agents may be used at a concentration of from InM to 1000 μM, preferably from 1 μM to 100 μM, more preferably from 1 μM to 10 μM.

In some embodiments of the methods provided herein, the subject having cancer shows decreased responsiveness to a CD47 inhibitor (e.g., is resistant or refractive to treatment with a CD47 inhibitor). In some embodiments, the subject is identified as having a decreased susceptibility (e.g., resistance or acquired resistance) to a monotherapy treatment of a CD47 inhibitor. In some embodiments, the subject is identified as having an increased susceptibility to a combination therapy treatment provided herein.

Also provided herein are methods of delaying resistance of a subject, or prolonging remission (e.g., complete remission or partial remission) of a subject, to a CD47 inhibitor as described herein. In some embodiments, the method of delaying resistance of the subject, or prolonging remission (e.g., complete remission or partial remission) of the subject, comprises administering a combination of a PI3K inhibitor (e.g., duvelisib, tenalisib, or IPI-549) and a CD47 inhibitor as described herein to the subject before the subject develops resistance to the CD47 inhibitor. In some embodiments, the method of delaying resistance of the subject, or prolonging remission (e.g., complete remission or partial remission) of the subject, comprises administering a combination of a PI3K inhibitor (e.g., Compound , tenalisib, or IPI-549) and a CD47 inhibitor described herein to the subject after the subject develops resistance to CD47 inhibitor.

In some embodiments, the subject is not resistant to a CD47 inhibitor as described herein. In some embodiments, the subject is not resistant to a PI3K Inhibitor as described herein. In some embodiments, the subject has previously been administered a PI3K inhibitor (e.g., duvelisib, tenalisib, or IPI-549) as a monotherapy or in combination with an agent other than a CD47 inhibitor as described herein. In some embodiments, the subject has previously been administered a CD47 inhibitor as described herein as a monotherapy or in combination with an agent other than a PI3K inhibitor as described herein. In some embodiments, the subject has a cancer, e.g., a cancer described herein. In some embodiments, in accordance with the method, resistance to reduction of CD47/SIRPα interaction is delayed compared to the time in which resistance generally develops when the subject is treated with a CD47 inhibitor alone as monotherapy. In some embodiments, the resistance is delayed by at least 2 weeks, e.g., at least 2 weeks, 4 weeks, 1 month, 2 months, 3 months, 4 months, 5 months, 6 months, 8 months, 10 months, 12 months, 1 year, 2 years, 4 years, 6 years, 8 years, or more. In some embodiments, in accordance with the method, remission (e.g., complete remission or partial remission) is prolonged compared to the time in which remission generally lasts when the subject is treated with any of the inhibitors alone as monotherapy. In some embodiments, remission (e.g., complete remission or partial remission) is prolonged by at least 2 weeks, e.g., at least 2 weeks, 4 weeks, 1 month, 2 months, 3 months, 4 months, 5 months, 6 months, 8 months, 10 months, 12 months, 1 year, 2 years, 4 years, 6 years, 8 years, or more.

Provided herein is also a method of improving the efficacy of a CD47 inhibitor, the method comprising, (a) administering an effective amount of PI3K inhibitor for a first period of time; (b) after the first period of time, administering to the subject a therapeutically effective amount of a combination therapy comprising the PI3K inhibitor in combination with a CD47 inhibitor for a second period of time; and optionally repeating steps (a) and (b) one or more times.

Provided herein is also a method of reducing a hematologic-related side effect and/or toxicity in a subject having cancer and receiving treatment with a CD47 inhibitor for the cancer, the method comprising administering to the subject a therapeutically effective amount of a PI3K inhibitor in combination with the CD47 inhibitor.

### Compositions and Methods

Provided herein are, at least in part, methods and compositions comprising a PI3K inhibitor, wherein the PI3K inhibitor can be any PI3K inhibitor as described herein below, including pharmaceutically acceptable forms thereof, and a CD47 inhibitor, wherein the CD47 inhibitor can be any CD47 inhibitor as described herein below, including pharmaceutically acceptable forms thereof.

As used herein, a “phosphoinositide 3-kinase (P13K) inhibitor” or “PI3K inhibitor” refers to an inhibitor of any PI3K (i.e., one or more isoforms). PI3Ks are members of a unique and conserved family of intracellular lipid kinases that phosphorylate the 3′-OH group on phosphatidylinositols or phosphoinositides. The PI3K family includes kinases with distinct substrate specificities, expression patterns, and modes of regulation (see, e.g., Katso et al., Annu. Rev. Cell I)ev. Biol. (2001) 1.7: 615 -675; Foster, F. M. et al., J. Cell Sci. (2003) 1 16: 3037-3040). The class I PI3Ks (e.g., p110α, p110β, p110γ, and p110δ) are typically activated by tyrosine kinases or G-protein coupled receptors to generate PIP3, which engages downstream mediators such as those in the AKT/PDK1 pathway, mTOR, the Tee family kinases, and the Rho family GTPases. The class II PI3Ks (e.g., PI3K-C2a, PI3K-C2β, PI3K-C2γ) and III PI3Ks (e.g., Vps34) play a key role in intracellular trafficking through the synthesis of PI(3)P, and PI(3,4)P2. Specific exemplary PI3K inhibitors are disclosed herein.

The class I PI3Ks comprise a p110 catalytic subunit and a regulatory adapter subunit. See, e.g., Cantrell, D. A. J. Cell Sci. (2001) 1 14: 1439-1445. Four isoforms of the p110 subunit (including PI3K-α (alpha), PI3K-β (beta), PI3K-γ (gamma), and PI3K-δ (delta) isoforms) have been implicated in various biological functions. Class I PI3K-α is involved, for example, in insulin signaling, and has been found to be mutated in solid tumors. Class I PI3K-β is involved, for example, in platelet activation and insulin signaling. Class I PI3K-γ plays a role in mast cell activation, innate immune function, and immune cell trafficking (chemokines). Class I PI3K-δ is involved, for example, in B-cell and T-cell activation and function and in Fe receptor signaling in mast cells. In some embodiments provided herein, the P13K inhibitor is a class I PI3K inhibitor. In some such embodiments, the PI3K inhibitor inhibits a PI3K-α (alpha), PI3K-γ (beta), PI3K-γ (gamma), or PI3K-δ (delta) isoform, or a combination thereof.

Downstream mediators of the PI3K signal transduction pathway include AKT and mammalian target of rapamycin (mTOR). (Manning et at., Cell (2007) 129: 1261-1274) AKT possesses a plckstrin homology (PH) domain that binds PIP3, leading to AKT kinase activation. AKT phosphorylates many substrates and is a central downstream effector of PI3K for diverse cellular responses. One important function of AKT is to augment the activity of mTOR, through phosphorylation of TSC2 and other mechanisms. mTOR is a serine-threonine kinase related to the lipid kinases of the PI3K family. (Lapiante et at, Cell (2012) 149: 274-293) mTOR has been implicated in a wide range of biological processes including cell growth, cell proliferation, cell motility and survival.

Disregulation of the mTOR pathway has been reported in various types of cancer, mTOR is a multifunctional kinase that integrates growth factor and nutrient signals to regulate protein translation, nutrient uptake, autophagy, and mitochondrial function.

PI3K-γ controls a critical switch between immune stimulation and suppression during inflammation and cancer. PI3Kγ signaling through Akt and mTor inhibits NFκB activation while stimulating C/EBPβ activation, thereby inducing a transcriptional program that promotes immune suppression during inflammation and tumor growth. By contrast, selective inactivation of macrophage PI3Kγ stimulates and prolongs NFκB activation and inhibits C/EBPβ activation, thus promoting an immunostimulatory transcriptional program that restores CD8⁺ T cell activation and cytotoxicity. PI3Kγ synergizes with checkpoint inhibitor therapy to promote tumor regression and increased survival in mouse models of cancer. In addition, PI3Kγ -directed, anti-inflammatory gene expression can predict survival probability in cancer patients (Kaneda et al. Nature (2016) 539: 437).

In some embodiments, provided herein are pharmaceutical compositions comprising a PI3K inhibitor, or a pharmaceutically acceptable form thereof, in combination with a CD47 inhibitor, or a pharmaceutically acceptable form thereof. In some embodiments, the combination is therapeutically effective. In some embodiments, the combination is synergistic, e.g. , has one or more synergistic effects, e.g., synergistic therapeutic effects.

Also provided herein are methods of treating, managing, or preventing a cancer in a subject comprising administering to the subject a PI3K inhibitor, or a pharmaceutically acceptable form thereof, in combination with a CD47 inhibitor, or a pharmaceutically acceptable form thereof. In some embodiments, the combination is therapeutically effective. In some embodiments, the combination is synergistic, e.g. , has one or more synergistic effects, e.g., synergistic therapeutic effects.

In some embodiments, the compositions and methods provided herein are utilized where a monotherapy of one of the therapeutic agents is becoming less effective due to drug resistance or where the relatively high dosage of monotherapy lead to undesirable side effects.

### PI3K Inhibitors

PI3K inhibitors that can be used in the compositions and methods provided herein include, but are not limited to, those described in, e.g., WO (19/(188990, WO2011/008302, WO 2010/036380, WO 2010/006086, WO 09/114870, WO 05/113556, and US 2011/0046165, the entirety of each incorporated herein by reference. Additional PI3K inhibitors that can be used in the compositions and methods provided herein include, but are not limited to, AMG-319, GSK 2126458 (2,4-Difluoro-N-{2-(methyloxy)-5-[4-(4-pyridazinyl)-6-quinolinyl]-3-pyridinyl}benzenesulfonamide), GSK 1059615 (5Z-[[4-(4-pyridinyl)-6-quinolinyl]methylene]-2,4-thiazolidinedione), GDC-0032 (4-[5,6-dihydro-2-[3-methyl-1-(1-methylethyl)-1H-1,2,4-triazol-5-yl]imidazo[1,2-d][1,4]benzoxazepin-9-yl]-α,α-dimethyl-1H-Pyrazole-1-acetamide), GDC-0980 ((S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one), GDC-0941 (2-(1H-indazol-4-yl)-6-yl)-6-((4-methylsulfonyl)piperazin-1-yl)methyl)-4-morpholinothieno[3,2-d]pyrimidine), XL147 (N-((3-(benzo[c][1,2,5]thiadiazol-5-ylamino)quinoxalin-2-yl)-4-methylbenzenesulfonamide), XL499, XL765 (SAR245409, N-[4-[[[3-[(3,5-dimethoxyphenyl)amino]-2-quinoxalinyl]amino]sulfonyl]phenyl]-3-methoxy-4-methyl-benzamide), PF-4691502 (2-amino-6-(6-methoxypyridin-3-yl)-4-methyl-8-[(1R,4R)-4-(2-hydroxyethoxy)cyclohexyl]-7H,8H-pyrido[2,3-d]pyrimidin-7-one), BKM 120 (buparlisib, 5-(2,6-dimorpholinopyrimidin-4-yl)-4-(tritluoromethyl)pyridin-2-amine), Idelalisib (CAL-101, GS 1101, (S)-2-(1-(9H-purin-6-ylamino)propyl)-5-fluoro-3-phenylquinazolin-4(3H)-one), CAL 263, SF1126 (3-[[2-[[5-[[amino(azaniumyl)methylidene]amino]-2-[[4-oxo-4-[4-(4-oxo-8-phenylchromen-2-yl)morpholin-4-ium-4-yl]oxybutanoyl]amino]pentanoyl]amino]acetyl]amino]- 4-(1-carboxylatopropylamino)-4-oxobutanoate), PX-866 (sonolisib, [(3aR,6E,9S,9aR,10R, 11aS) 6-[[bis(prop-2-enyl)amino]methylidene]-5-hydroxy-9-(methoxymethyl)-9a, 11a-dimethyl-1,4,7-trioxo-2,3,3a,9, 10, 11-hexahydroindeno[4,5-h]isochromen-10-yl]acetate), BEZ235 (2-methyl-2-(4-(3-methyl-2-oxo-8-(quinolin-3-yl)-2,3-dihydroimidazo[4,5-c]quinolin-1-yl)phenyl)propanenitrile), GS9820 (CAL-120, (S)-2-(1- ((9H-purin-6-yl)amino)ethyl)-6-fluoro-3-phenylquinazolin-4(3H)-one), BYL719 ((2S)-1,2-Pyrrolidinedicarboxamide, N1-[4-methyl-5-[2-(2,2,2-trifluoro-1,1-dimethylethyl)-4-pyridinyl]-2-thiazolyl]), RP6503, tenalisib (RP6530, (S)-2-(1-((7H-purin-6-yl)amino)propyl)-3-(3-fluorophenyl)-4H-chromen-4-one), TGR1202 (((S)-2-(1-(4-amino-3-(3-fluoro-4-isopropoxyphenyl)-1H-pyrazolo [3,4-d]pyrimidin-1-yl)ethyl)-6-fluoro-3-(3-fluorophenyl)-4H-chromen-4-one)), INK1117 (MLN-1117), BAY 80-6946 (2-amino-N-(7-methoxy-8-(3-morpholinopropoxy)-2,3-dihydroimidazo[1,2-c]quinazolin-5-yl)pyrimidine-5-carboxamide), IC87114 (2-(((6-amino-9H-purin-9-yl)methyl)-5-methyl-3-o-tolylquinazolin-4(3H)-one), Palomid 529 (3-(4-methoxybenzyloxy)-8-(1-hydroxyethyl)-2-methoxy-6H-benzo[c]chromen-6-one), ZSTK474 (2-(difluoromethyl)-1-(4,6-dimorpholino-1,3,5-triazin-2-yl)-1H-benzo [d]imidazole), PWT33597, TG 100-115 (6,7-Bis(3-hydroxyphenyl)pteridine-2,4-diamine), GNE-477 (5-[7-methyl-4-(morpholin-4-yl)-6-[(4-methylsulfonylpiperazin-1-yl)methyl]thieno[3,2-d]pyrimidin-2-yl]pyrimidin-2-amine), CUDC-907 (N-hydroxy-2-(((2-(6-methoxypyridin-3-yl)-4-morpholinothieno[3,2-d]pyrimidin-6-yl)methyl)(methyl)amino)pyrimidine-5-carboxamide), AEZS-136, BGT-226 (8-(6-methoxypyridin-3-yl)-3-methyl-1-(4-(piperazin-1-yl)-3-(trifluoromemyl)phenyl)-1H-imidazo[4,5-c]quinolin-2(3H)-one maleic acid), PF-05212384 (1-(4-(4-(dimethylamino)piperidine-1-carbonyl)phenyl)-3-(4-(4,6-dimorpholino-1,3,5-triazin-2-yl)phenyl)urea), LY3023414, PI-103 (3-[4-(4-morpholinyl)pyrido[3′,2′:4,5]furo[3,2-d]pyrimidin-2-yl]-phenol), INCB040093, CAL-130 ((S)-2-(1-((2-amino-9H-purin-6-yl)amino)ethyl)-5-methyl-3-(o-tolyl)quinazolin-4(3H)-one), LY294002 (2-Morpholin-4-yl-8-phenylchromen-4-one), wortmannin, AS252424 (5-[1-[5-(4-Fluoro-2-hydroxy-phenyl)-furan-2-yl]-meth-(Z)-ylidene]-thiazolidine-2,4-dione), IPI-549 (2-amino-N-[(1S)-1-[8-[2-(1-methylpyrazol-4-yl)ethynyl]-1-oxo-2-phenylisoquinolin-3-yl]ethyl]pyrazolo[1,5-a]pyrimidine-3-carboxamide), CZC24832 (5-(2-Amino-8-fluoro-[1,2,4]triazolo[1,5-a]pyridin-6-yl)-N-tert-butylpyridine-3-sulfonamide), duvelisib (INK-1197, IPI-145, Copiktra™, (S)-3-(1-((9H-purin-6-yl)amino)ethyl)-8-chloro-2-phenylisoquinolin-1(2H)-one), or AS-604850 (5-(2,2-Difluoro-benzo[1,3]dioxol-5-ylmethylene)-thiazolidine-2,4-dione).

In some embodiments, a PI3K inhibitor is a compound that inhibits one or more PI3K isoforms, e.g., alpha, beta, delta, or gamma isoform. In some embodiments, a PI3K inhibitor is a compound that inhibits one or more PI3K isoforms with an IC₅₀ of less than about 1000 nM, less than about 900 nM, less than about 800 nM, less than about 700 nM, less than about 600 nM, less than about 500 nM, less than about 400 nM, less than about 300 nM, less than about 200 nM, less than about 100 nM, less than about 75 nM, less than about 50 nM, less than about 25 nM, less than about 20 nM, less than about 15 nM, less than about 10 nM, less than about 10 nM, less than about 5 nM, or less than about 1 nM.

In some embodiments, the inhibitor is a compound that inhibits alpha, beta, delta and gamma isoforms of Pi3K. In another embodiment, the PI3K inhibitor is a compound that inhibits beta, delta, and gamma isoforms of PI3K. In another embodiment, the PI3K inhibitor is a compound that inhibits the delta and gamma isoforms of PI3K.

In some embodiments, the PI3K inhibitor is a PI3K isoform selective inhibitor. In some embodiments, the PI3K inhibitor is a PI3K alpha selective inhibitor. In another embodiment, the PI3K inhibitor is a PI3K beta selective inhibitor.

In some embodiments, the PI3K inhibitor is a PI3K gamma selective inhibitor. In some embodiments, the PI3K gamma selective inhibitor selectively inhibits PI3K gamma isoform over PI3K delta isoform. In some embodiments, the PI3K gamma selective inhibitor has a delta/gamma selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In some embodiments, the PI3K gamma selective inhibitor has a delta/gamma selectivity ratio in the range of from greater than 1 to about 5, from about 5 to about 10, from about 10 to about 50, from about 50 to about 850, or greater than about 850. In some embodiments, the delta/gamma selectivity ratio is determined by dividing the inhibitor's IC₅₀ against PI3K delta isoform by the inhibitor's IC₅₀ against PI3K gamma isoform.

In some embodiments, the PI3K inhibitor is a PI3K gamma selective inhibitor. In some embodiments, the PI3K gamma selective inhibitor selectively inhibits PI3K gamma isoform over PI3K alpha isoform. In some embodiments, the PI3K gamma selective inhibitor has an alpha/gamma selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In some embodiments, the PI3K gamma selective inhibitor has an alpha/gamma selectivity ratio in the range of from greater than 1 to about from about 5 to about 10, from about 10 to about 50, from about 50 to about 850, or greater than about 850. In some embodiments, the alpha/gamma selectivity ratio is determined by dividing the inhibitor's IC₅₀ against PI3K alpha isoform by the inhibitor's IC₅₀ against PI3K gamma isoform.

In some embodiments, the PI3K inhibitor is a PI3K gamma selective inhibitor. In some embodiments, the PI3K gamma selective inhibitor selectively inhibits PI3K gamma isoform over PI3K beta isoform, in some embodiments, the PI3K gamma selective inhibitor has a beta/gamma selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In some embodiments, the PI3K gamma selective inhibitor has a beta/gamma selectivity ratio in the range of from greater than 1 to about 5, from about 5 to about 10, from about 10 to about 50, from about 50 to about 850, or greater than about 850. In some embodiments, the beta/gamma selectivity ratio is determined by dividing the inhibitor's IC₅₀ against PI3K beta isoform by the inhibitor's IC₅₀ against PI3K gamma isoform.

In some embodiments, the PI3K inhibitor is selective for both gamma and delta. In some embodiments, the PI3K gamma and delta selective inhibitor selectively inhibits PI3K gamma and delta isoforms over PI3K beta isoform. In some embodiments, the PI3K gamma and delta selective inhibitor has a beta/delta selectivity ratio of greater than 1. greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000 and a beta/gamma selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In some embodiments, the PI3K gamma and delta selective inhibitor has a beta/delta selectivity ratio in the range of from greater than 1 to about 5, from about 5 to about 10, from about 10 to about 50, from about 50 to about 850, or greater than about 850 and a beta/gamma selectivity ratio in the range of from greater than 1 to about 5, from about 5 to about 10, from about 10 to about 50, from about 50 to about 850, or greater than about 850. In some embodiments, the beta/delta selectivity ratio is determined by dividing the inhibitor's IC₅₀ against PI3K beta isoform by the inhibitor's IC₅₀ against PI3K delta isoform and the beta/gamma selectivity ratio is determined by dividing the inhibitor's IC₅₀ against PI3K beta isoform by the inhibitor's IC₅₀ against PI3K gamma isoform.

PI3K gamma inhibitors that can be used in the compositions and methods provided herein include, but are not limited to, IPI-549 (2-amino-N-[(1S)-1-[8-[2-(1-methylpyrazol-4-yl)ethynyl]-1-oxo-2-phenylisoquinolin-3-yl]ethyl]pyrazolo[1,5-a]pyrimidine-3-carboxamide), CZC24832 (5-(2-Amino-8-fluoro-[1,2,4]triazolo[1,5-a]pyridin-6-yl)-N-tert-butylpyridine-3-sulfonamide), AS252424 (5-[1-[5-(4-Fluoro-2-hydroxy-phenyl)-furan-2-yl]-meth-(Z)-ylidene]-thiazolidine-2,4-dione), or AS-604850 (5-(2,2-Difluoro-benzo[1,3]dioxo1-5-ylmethylene)-thiazolidine-2,4-dione. In some embodiments, the PI3K gamma inhibitor is IPI-549.

In some embodiments, the PI3K inhibitor is a PI3K delta/gamma dual inhibitor. In some embodiments, the PI3K delta/gamma dual inhibitor has an IC₅₀ value against PI3K alpha that is at least 5×, 10×, 20×, 50×, 100×, 200×, 500×, or 1000× higher than its IC₅₀ values against delta and gamma.

### CD47 Inhibitors

CD47 is an integrin-associated receptor protein that is expressed on a broad range of cell types, including red blood cells. CD47 interacts with signal regulatory protein-α (SIPRα), thrombospondin (TSP)-1 and -2 to mediate various cellular functions. In particular, CD47 signaling through SIPRαinhibits the phagocytosis of CD47-expressing target cells by SIPRα, expressing macrophages. CD47 inhibitors block the interaction of CD47 and SIPRα and promote the phagocytosis of tumor cells by macrophages, and also by monocytes and neutrophils. (Kim, D. et al., Leukemia (2012) 26: 2538-2545).

SIRPα is a membrane protein of the immunoglobulin superfamily that is particularly abundant in the myeloid-lineage hematopoietic cells such as macrophages and dendritic cells. The ligation of SIRPα on phagocytes by CD47 expressed on a neighboring cell results in phosphorylation of SIRPα cytoplasmic immunoreceptor tyrosine-based inhibition (ITIM) motifs, leading to the recruitment of SHP-1 and SHP-2 phosphatases. One resulting downstream effect is the prevention of myosin-IIA accumulation at the phagocytic synapse and consequently inhibition of phagocytosis. Thus, CD47-SIRPα interaction functions as a negative immune checkpoint to send a “don't eat me” signal to ensure that healthy autologous cells are not inappropriately phagocytosed. (Liu, X., et al. Journal of Hematology and Oncology (2017) 10:12).

Cancer cells including acute myelogenous leukemias, lymphomas, and various solid cancers express CD47 at a high level and can be phagocytosed by macrophages in the presence of blocking antibodies to CD47. Blocking the CD47-SIPRα interaction with anti-CD47 monoclonal antibodies has proven effective in inducing the phagocytosis of tumor cells in vitro and inhibiting the growth of various hematological and solid tumors ire vivo (Kim, D, et al., Leukemia (2012) 26: 2538-2545). The blockade of CD47 is not only limited to anti-CD47 antibodies but can also include recombinant fusion proteins consisting of the CD47 binding domain of human SIRPα linked to the Fe region of human IgG1, which is designed to both 1) block the CD47 “don't eat me” signal, and 2) engage macrophage Fcγ receptors with IgG1 Fc to enhance phagocytosis and antitumor activity (Ansell, S. et al. Blood (2016) 128: 1812).

As used herein, the term “CD47 inhibitor” refers to any agent that reduces the binding of CD47 (e.g., on a target cell) to SIRPα (e.g., on a phagocytic cell). Examples of suitable CD47 inhibitors include SIRPα reagents, including without limitation high affinity SIRPα polypeptides, anti-SIRPα antibodies, soluble CD47 polypeptides, and anti-CD47 antibodies, antibody fragments, peptides, small molecules, peptidomimetics, and the like. In some embodiments, a suitable CD47 inhibitor (e.g., an anti-CD47 antibody, a SIPRα reagent, etc.) specifically binds to CD47 to reduce the binding of CD47 to SIPRα. In some embodiments, a suitable CD47 inhibitor (e.g., an anti-SIRPα antibody, a soluble CD47 polypeptide, etc.) specifically binds SIRPα to reduce the binding of CD47 to SIRPα. In some embodiments, a suitable CD47 inhibitor that binds SIRPα does not activate SIRPα (e.g., in the SIRPα-expressing phagocytic cell). In some embodiments, the CD47 inhibitor does not activate CD47 upon binding. When CD47 is activated, a process akin to apoptosis (i.e., programmed cell death) may occur (Manna and Frazier Cancer Research (2004) 64:1026-1036). Thus, in some embodiments, the anti-CD47 agent does not directly induce cell death of a CD47-expressing cell.

A SIRPα reagent comprises the portion of SIRPα that is sufficient to bind CD47 at a recognizable affinity, which normally lies between the signal sequence and the transmembrane domain, or a fragment thereof that retains the binding activity. A suitable SIRPα reagent reduces (e.g., blocks, prevents, etc.) the interaction between the native proteins SIRPα and CD47. The SIRPα reagent will usually comprise at least the dl domain of SIRPα. In some embodiments, a SIRPα reagent is a fusion protein, e.g., fused in frame with a second polypeptide (e.g., TTI-621 (Trillium Therapeutics), see Petrova, P. S. et al. Clin. Cancer Res. (2017) 23(4): 1068-1079 and ALX-148 (Alexo Therapeutics), see Kauder, S. E. et al. Blood (2017), 130:1_suppl: 112). In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region (e.g., TTI-621). The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

In some embodiments, a subject CD47 inhibitor is a “high affinity SIRPα reagent”, which includes SIRPα-derived polypeptides and analogs thereof. High affinity SIRPα reagents are described in international application PCT/US13/21937, which is hereby specifically incorporated by reference. High affinity SIRPα reagents are variants of the native SIRPα protein. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

A high affinity SIRPα reagent comprises the portion of SIRPα that is sufficient to bind CD47 at a recognizable affinity, e.g., high affinity, which normally lies between the signal sequence and the transmembrane domain, or a fragment thereof that retains the binding activity. The high affinity SIRPα reagent will usually comprise at least the dl domain of SIRPα with modified amino acid residues to increase affinity. In some embodiments, a SIRPα variant of the present invention is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules. The amino acid changes that provide for increased affinity are localized in the dl domain, and thus high affinity SIRPα reagents comprise a dl domain of human SIRPα, with at least one amino acid change relative to the wild-type sequence within the dl domain. Such a high affinity SIRPα reagent optionally comprises additional amino acid sequences, for example antibody Fc sequences; portions of the wild-type human SIRPα protein other than the dl domain, including without limitation residues 150 to 374 of the native protein or fragments thereof, usually fragments contiguous with the dl domain; and the like. High affinity SIRPα reagents may be monomeric or multimeric, i.e. dimer, trimer, tetramer, etc.

In some embodiments, a suitable CD47 inhibitor is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activated CD47 upon binding. Non-limiting examples of suitable antibodies include B6H12, CC-90002 (Celgene) (see Zheng, B. et al. Cancer Res. (2017) 77:13_suppl, doi: 10.1158/1538-7445.AM2017-2009), Hu5F9-G4 (Forty Seven) (see Advani, R. H. et al., J. Clin. Oncol. 36, 2018 (suppl; abstr 7504) and Agoram, B. et al. J. Clin. Oncol. 36, 2018 (suppl; abstr 2525)), TI-061 (Tioma Therapeutics) (see Evans J. T. R. et al. J. Clin. Oncol. (2017) 35:15_suppl, doi: 10.1200/JCO.2017.35.15_suppl.TPS3109), SRF-213 (Surface Oncology) (see Holland P. M. et al. Blood (2016) 128 (22): 1843), 13H3 Ab (SEQ ID NO.: 31 and SEQ ID NO.: 32 as disclosed in International Publication No. WO 2018/075857), and 1F8 Ab (SEQ ID NO.: 3 and SEQ ID NO.: 4 as disclosed in International Publication WO2018/075857). The sequences and all related description are incorporated by reference. Suitable anti-CD47 antibodies include fully human, humanized or chimeric versions of such antibodies. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

In some embodiments, a suitable CD47 inhibitor is an antibody that specifically binds SIRPα (i.e., an anti-SIRPα antibody) and reduces the interaction between CD47 on one cell and SIRPα on another cell. Suitable anti-SIRPα antibodies can bind SIRPα without activating or stimulating signaling through SIRPα, because activation of SIRPα would inhibit phagocytosis. Instead, suitable anti-SIRPα antibodies facilitate the phagocytosis of target cells. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

In some embodiments, a subject CD47 inhibitor is a soluble CD47 polypeptide that specifically binds SIRPα and reduces the interaction between CD47 on one cell and SIRPα on another cell. A suitable soluble CD47 polypeptide can bind SIRPα without activating or stimulating signaling through SIRPα. Suitable soluble CD47 polypeptides facilitate the phagocytosis of target cells. Thus, a suitable soluble CD47 polypeptide specifically binds SIRPα without activating/stimulating enough of a signaling response to inhibit phagocytosis.

In some embodiments, a suitable soluble CD47 polypeptide can be a fusion protein. Suitable soluble CD47 polypeptides also include any peptide or peptide fragment comprising variant or naturally existing CD47 sequences (e.g., extracellular domain sequences or extracellular domain variants) that can specifically bind SIRPα and inhibit the interaction between CD47 and SIRPα without stimulating enough SIRPα activity to inhibit phagocytosis.

The terms “phagocytic cells” and “phagocytes” are used interchangeably herein to refer to a cell that is capable of phagocytosis, i.e., engulfing a large particulate mass, for example from about 0.1 .mu.m in diameter up to about 2 mm or about 1 mm in diameter; from about 0.5 .mu.m in diameter in to about 1 mm in diameter, etc, particularly including up to the size of a mammalian cell, e.g. a tumor cell. Phagocytosis in this context is defined by the engulfment of cells, pathogens, and various particles by surrounding it with the effector cell membrane.

There are several categories of phagocytes: macrophages; mononuclear cells (histiocytes and monocytes); polymorphonuclear leukocytes; (neutrophils) and dendritic cells. Macrophages are of particular interest. Phagocytosis-associated cell responses include immunomodulatory responses like the generation and release of pro-inflammatory and anti-inflammatory mediators, and also cell responses of destructive nature such as the respiratory burst, and the release of toxic and microbicidal molecules by degranulation. Professional phagocytes are capable of recognizing a wide variety of phagocytic targets, and of ingesting them at a higher rate than non-phagocytic cells.

The term “antibody” as used herein refers to any polypeptide chain-containing molecular structure with a specific shape that fits to and recognizes an epitope, where one or more non-covalent binding interactions stabilize the complex between the molecular structure and the epitope. Antibodies utilized in the disclosure are monoclonal antibodies though they may also include polyclonal antibodies.

### Formulations

The formulations or compositions described herein can include a PI3K inhibitor (e.g., one or more PI3K inhibitors as described herein) and a CD47 inhibitor (e.g., one or more CD47 inhibitors as described herein). In some embodiments, the PI3K inhibitor (e.g., one or more PI3K inhibitors as described herein) and the CD47 inhibitor (e.g., one or more CD47 inhibitors as described herein) are included in the same dosage form. In some embodiments, the PI3K inhibitor (e.g., one or more PI3K inhibitors as described herein) and the CD47 (e.g., one or more CD47 inhibitors as described herein) inhibitor are included in separate dosage forms. In some embodiments, the formulations or compositions described herein (e.g., a PI3K inhibitor in combination with a CD47 inhibitor) further comprise an opsonizing antibody. In some embodiments, the opsonizing antibody is an anti-CD20 antibody (e.g., rituximab, obinutuzumab, ofatumumab), an anti-EGFR antibody (e.g., cetuximab, panitumumab), an anti-HER2 antibody (e.g., trastuzumab, pertuzumab), an anti-CD38 antibody (e.g., daratumumab, isatuximab), an anti-CD19 antibody (e.g., tafasitamab), an anti-CD22 antibody (e.g., moxetumomab pasudotox), an anti-CCR4 antibody (e.g., mogamulizumab), or any other opsonizing antibody.

Pharmaceutical compositions may be specially formulated for administration in solid or liquid form, including those adapted for the following: oral administration, for example, drenches (aqueous or non-aqueous solutions or suspensions), tablets (e.g., those targeted for buccal, sublingual, and systemic absorption), capsules, boluses, powders, granules, pastes for application to the tongue, and intraduodenal routes; parenteral administration, including intravenous, intraarterial, subcutaneous, intramuscular, intravascular, intraperitoneal or infusion as, for example, a sterile solution or suspension, or sustained-release formulation; topical application, for example, as a cream, ointment, or a controlled-release patch or spray applied to the skin; intravaginally or intrarectally, for example, as a pessary, cream, stent or foam; sublingually; ocularly; pulmonarily; local delivery by catheter or stent; intrathecally, or nasally.

The amount of P13K inhibitor and/or CD47 inhibitor administered and the timing of PI3K inhibitor and/or CD47 inhibitor administration will depend on the type (species, gender, age, weight, etc.) and condition of the patient being treated, the severity of the disease or condition being treated, and on the route of administration. For example, small molecule PI3K inhibitors or CD47 inhibitors can be administered to a patient in doses ranging from 0.001 to 100 mg/kg of body weight per day or per week in single or divided doses, or by continuous infusion. Antibody-based P13K inhibitors or CD47 inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

Examples of suitable aqueous and nonaqueous carriers which may be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions can also contain adjuvants such as preservatives, wetting agents, emulsifying agents, dispersing agents, lubricants, and/or antioxidants. Prevention of the action of microorganisms upon the compounds described herein may be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Methods of preparing these formulations or compositions include the step of bringing into association a compound described herein and/or the chemotherapeutic with the carrier and, optionally, one or more accessory ingredients. In general, the formulations are prepared by uniformly and intimately bringing into association a compound as disclosed herein with liquid carriers, or finely divided solid carriers, or both, and then, if necessary, shaping the product.

Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Twelfth Edition, McGraw Hill, 2011 ; Goodman and Gilman, eds., The Pharmacological Basis of Therapeutics, Tenth Edition, McGraw Hill, 2001; Remingions Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety. Except insofar as any conventional excipient medium is incompatible with the compounds provided herein, such as by producing any undesirable biological effect or otherwise interacting in a deleterious manner with any other component(s) of the pharmaceutically acceptable composition, the excipient's use is contemplated to be within the scope of this disclosure.

In some embodiments, the concentration of the PI3K inhibitor (e.g., duvelisib, tenalisib, or IPI-549) or CD47 inhibitor provided a pharmaceutical composition disclosed herein or administered in a method disclosed herein is less than about 100%, about 90%, about 80%, about 70%, about 60%, about 50%, about 40%, about 30%, about 20%, about 19%, about 18%, about 17%, about 16%, about 15%, about 14%, about 13%, about 12%, about 1 1%, about 10%, about 9%, about 8%, about 7%, about 6%, about 5%, about 4%, about 3%, about 2%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001%, w/w, w/v or v/v.

In some embodiments, the concentration of the PI3K inhibitor (e.g., duvelisib, tenalisib, or IPI-549) or CD47 inhibitor provided a pharmaceutical composition disclosed herein or administered in a method disclosed herein is greater than about 90%, about 80%, about 70%, about 60%, about 50%, about 40%, about 30%, about 20%, about 19.75%, about 19.50%, about 19.25%, about 19%, about 18.75%, about 18.50%, about 18.25%, about 18%, about 17.75%, about 17.50%, about 17.25%, about 17%, about 16.75%, about 16.50%, about 16.25%, about 16%, about 15.75%, about 15.50%, about 15.25%, about 15%, about 14.75%, about 14.50%, about 14.25%, about 14%, about 13.75%, about 13.50%, about 13.25%, about 13%, about 12.75%, about 12.50%, about 12.25%, about 12%, about 1.75%, about 11.50%, about 1 1.25%, about 1 1%, about 10.75%, about 10.50%, about 10.25%, about 10%, about 9.75%, about 9.50%, about 9.25%, about 9%, about 8.75%, about 8.50%, about 8.25%, about 8%, about 7.75%, about 7.50%, about 7.25%, about 7%, about 6.75%, about 6.50%, about 6.25%, about 6%, about 5.75%, about 5.50%, about 5.25%, about 5%%, about 4.75%, about 4.50%, about 4.25%, about 4%, about 3.75%, about 3.50%, about 3.25%, about 3%, about 2.75%, about 2.50%, about 2.25%, about 2%, about 1.75%, about 1.50%, about 1.25%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001%, w/w, w/v, or v/v.

In some embodiments, the concentration of the PI3K inhibitor (e.g., duvelisib, tenalisib, or IPI-549) or CD47 inhibitor provided a pharmaceutical composition disclosed herein or administered in a method disclosed herein is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, or approximately 1% to approximately 10%, w/w, w/v or v/v.

In some embodiments, the concentration of the PI3K inhibitor (e.g., duvelisib, tenalisib, or IPI-549) or CD47 inhibitor provided a pharmaceutical composition disclosed herein or administered in a method disclosed herein is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, or approximately 0.1% to approximately 0.9%, w/w, w/v or v/v.

In some embodiments, the concentration of the PI3K inhibitor (e.g., duvelisib, tenalisib, or IPI-549) or CD47 inhibitor provided a pharmaceutical composition disclosed herein or administered in a method disclosed herein is equal to or less than about 10 g, about 9.5 g, about 9.0 g, about 8.5 g, about 8.0 g, about 7.5 g, about 7.0 g, about 6.5 g, about 6.0 g, about 5.5 g, about 5.0 g, about 4.5 g, about 4.0 g, about 3.5 g, about 3.0 g, about 2.5 g, about 2.0 g, about 1.5 g, about 1.0 g, about 0.95 g, about 0.9 g, about 0.85 g, about 0.8 g, about 0.75 g, about 0.7 g, about 0.65 g, about 0.6 g, about 0.55 g, about 0,5 g, about 0.45 g, about 0.4 g, about 0.35 g, about 0.3 g, about 0.25 g, about 0.2 g, about 0.15 g, about 0.1 g, about 0.09 g, about 0.08 g, about 0.07 g, about 0.06 g, about 0.05 g, about 0.04 g, about 0.03 g, about 0.02 g, about 0.01 g, about 0.009 g, about 0.008 g, about 0.007 g, about 0.006 g, about 0.005 g, about 0.004 g, about 0.003 g, about 0.002 g, about 0.001 g, about 0.0009 g, about 0.0008 g, about 0.0007 g, about 0.0006 g, about 0.0005 g, about 0,0004 g, about 0.0003 g, about 0.0002 g, or about 0.0001 g.

In some embodiments, the concentration of the PI3K inhibitor (e.g., duvelisib, tenalisib, or IPI-549) or CD47 inhibitor provided a pharmaceutical composition disclosed herein or administered in a method disclosed herein is more than about 0.0001 g, about 0.0002 g, about 0.0003 g, about 0.0004 g, about 0.0005 g, about 0.0006 g, about 0.0007 g, about 0.0008 g, about 0.0009 g, about 0.001 g, about 0.0015 g, about 0.002 g, about 0.0025 g, about 0.003 g, about 0.0035 g, about 0.004 g, about 0.0045 g, about 0.005 g, about 0.0055 g, about 0.006 g, about 0.0065 g, about 0.007 g, about 0.0075 g, about 0.008 g, about 0.0085 g, about 0,009 2, about 0.0095 g, about 0.01 g, about 0,015 2, about 0.02 g, about 0.025 g, about 0.03 g, about 0.035 g, about 0.04 g, about 0.045 g, about 0.05 g, about 0.055 g, about 0.06 g, about 0.065 2, about 0.07 g, about 0.075 g, about 0.08 g, about 0.085 g, about 0.09 g, about 0.095 g, about 0.1 g, about 0.15 about 0.2 g, about 0.25 g, about 0.3 g, about 0.35 g, about 0.4 g, about 0.45 g, about 0.5 g, about 0.55 g, about 0.6 g, about 0.65 g, about 0,7 g, about 0.75 g, about 0.8 g, about 0.85 g, about 0.9 g, about 0.95 g, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g.

### Formulations for Oral Administration

In some embodiments of the methods described herein, PI3K inhibitor (e.g., one or more PI3K inhibitors) and/or CD47 inhibitor (e.g., one or more CD47 inhibitors as described herein) is administered orally. In certain embodiments of the compositions described herein, PI3K inhibitor (e.g., duvelisib, tenalisib, or IPI-549) and/or CD47 inhibitor (e.g., one or more CD47 inhibitors as described herein) is formulated for oral administration. Some embodiments pertaining to such methods and compositions include the following.

In some embodiments, provided herein are pharmaceutical compositions for oral administration containing a compound as disclosed herein, and a pharmaceutical excipient suitable for oral administration. In some embodiments, provided herein are pharmaceutical compositions for oral administration containing: (i) an effective amount of one or more PI3K inhibitor; optionally (ii) an effective amount of one or more CD47 inhibitor; and (iii) one or more pharmaceutical excipients suitable for oral administration.

In some embodiments, the pharmaceutical composition can be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more ingredients. In general, the pharmaceutical compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The present disclosure further encompasses anhydrous pharmaceutical compositions and dosage forms comprising an active ingredient, since water can facilitate the degradation of some compounds. For example, water can be added (e.g., about 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. For example, pharmaceutical compositions and dosage forms which contain lactose can be made anhydrous if substantial contact with moisture and/or humidity during manufacturing, packaging, and/or storage is expected. An anhydrous pharmaceutical composition can be prepared and stored such that its anhydrous nature is maintained. Accordingly, anhydrous pharmaceutical compositions can be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the pharmaceutical compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. In some embodiments, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre -gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre -gelatinized starch, and mixtures thereof.

Disintegrants can be used in the pharmaceutical compositions as provided herein to provide tablets that disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active ingredient(s) can be used to form the dosage forms of the compounds disclosed herein. The amount of disintegrant used can vary based upon the type of formulation and mode of administration, and can be readily discernible to those of ordinary skill in the art. About 0.5 to about 15 weight percent of disintegrant, or about 1 to about 5 weight percent of disintegrant, can be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein can be combined with various sweetening or flavoring agents, coloring matter or dyes and, for example, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms include, but are not limited to, hydrophilic surfactants, lipophilic surfactants, and mixtures thereof. That is, a mixture of hydrophilic surfactants can be employed, a mixture of lipophilic surfactants can be employed, or a mixture of at least one hydrophilic surfactant and at least one lipophilic surfactant can be employed.

A suitable hydrophilic surfactant can generally have an HLB value of at least about 10, while suitable lipophilic surfactants can generally have an HLB value of or less than about 10. An empirical parameter used to characterize the relative hydrophilicity and hydrophobicity of non-ionic amphiphilic compounds is the hydrophilic -lipophilic balance (“HLB” value), Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants can be either ionic or non-ionic, Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants can be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants can include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of triglycerides, vegetable oils, and hydrogenated vegetable oils. The polyol can be glycerol, ethylene glycol, polyethylene glycol, sorbitol, propylene glycol, pentaerythritol, or a saccharide.

Other hydrophilic-non-ionic surfactants include, without limitation, PEG-10 laurate,PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG- 15 stearate, PEG-32 distearate, PEG-40 stearate, PEG- 100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of glycerides, vegetable oils, hydrogenated vegetable oils, fatty acids and sterols; oil-soluble vitamins/vitamin derivatives; and mixtures thereof. Within this group, non-limiting examples of lipophilic surfactants include glycerol fatty acid esters, propylene glycol fatty acid esters, and mixtures thereof, or are hydrophobic transesterification products of a polyol with at least one member of vegetable oils, hydrogenated vegetable oils, and triglycerides.

In some embodiments, the pharmaceutical composition can include a solubilizer to ensure good solubilization and/or dissolution of a compound as provided herein and to minimize precipitation of the compound. This can be especially important for pharmaceutical compositions for non-oral use, e.g., pharmaceutical compositions for injection. A solubilizer can also be added to increase the solubility of the hydrophilic drug and/or other components, such as surfactants, or to maintain the pharmaceutical composition as a stable or homogeneous solution or dispersion. Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ϵ-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ϵ-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers can also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. In some embodiments, solubilizers include sorbitol, glycerol, triacetin, ethyl alcohol, PEG-400, glycofurol and propylene glycol.

The amount of solubilizer that can be included is not particularly limited. The amount of a given solubilizer can be limited to a bioacceptable amount, which can be readily determined by one of skill in the art. In some circumstances, it can be advantageous to include amounts of solubilizers far in excess of bioacceptable amounts, for example to maximize the concentration of the drug, with excess solubilizer removed prior to providing the pharmaceutical composition to a subject using conventional techniques, such as distillation or evaporation. Thus, if present, the solubilizer can be in a weight ratio of about 10%, 25%, 50%, 100%, or up to about 200% by weight, based on the combined weight of the drug, and other excipients. If desired, very small amounts of solubilizer can also be used, such as about 5%, 2%, 1% or even less. Typically, the solubilizer can be present in an amount of about 1% to about 100%, more typically about 5% to about 25% by weight.

The pharmaceutical composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, oils, odorants, ,pacifiers, suspending agents, binders, fillers, plasticizers, lubricants, and mixtures thereof.

Exemplary preservatives can include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassiurn edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 15, Germaben II, Neolone, Kathon, and Euxyl. In certain embodiments, the preservative is an anti-oxidant. In other embodiments, the preservative is a chelating agent.

Exemplary oils include, but are not limited to, almond, apricot kernel, avocado, babassu, bergamot, black current seed, borage, carie, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam, seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In addition, an acid or a base can be incorporated into the pharmaceutical composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluene sulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Examples can include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, glucosic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluene sulfonic acid, uric acid and the like.

### Formulations for Parenteral Administration

In some embodiments of the methods described herein, PI3K inhibitor (e.g., one or more PI3K inhibitors) and/or CD47 inhibitor (e.g., one or more CD47 inhibitors as described herein) is administered parenterally. In certain embodiments of the compositions described herein, PI3K inhibitor (e.g., duvelisib, tenalisib, or IPI-549) and/or CD47 inhibitor (e.g., one or more CD47 inhibitors as described herein) is formulated for parenteral administration. Some embodiments pertaining to such methods and compositions include the following.

In some embodiments, provided herein are pharmaceutical compositions for parenteral administration containing a compound as disclosed herein, and a pharmaceutical excipient suitable for parenteral administration. In some embodiments, provided herein are pharmaceutical compositions for parenteral administration containing: (i) an effective amount of one or more PI3K inhibitor; optionally (ii) an effective amount of one or more CD47 inhibitor; and (iii) one or more pharmaceutical excipients suitable for parenteral administration. The forms in which the disclosed pharmaceutical compositions can be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils can also be employed.

Aqueous solutions in saline are also conventionally used for injection. Ethanol, glycerol, propylene glycol, liquid polyethylene glycol, and the like (and suitable mixtures thereof), cyclodextrin derivatives, and vegetable oils can also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound as disclosed herein in the required amount in the appropriate solvent with various other ingredients as enumerated above, as appropriate, followed by filtered sterilization. Generally, dispersions are prepared by incorporating the various sterilized active ingredients into a sterile vehicle which contains the basic dispersion medium and the appropriate other ingredients from those enumerated above. In the case of sterile powders for the preparation of sterile injectable solutions, certain methods of preparation are vacuum-drying and freeze-drying techniques which yield a powder of the active ingredient plus any additional ingredient from a previously sterile-filtered solution thereof.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Injectable compositions can contain from about 0.1 to about 5% w/w of a compound as disclosed herein.

### Dosage

The PI3K inhibitor (e.g., duvelisib, tenalisib, or IPI-549) or CD47 inhibitor may be delivered in the form of pharmaceutically acceptable compositions. In certain embodiments, the pharmaceutical compositions comprise the PI3K inhibitor (e.g., duvelisib, tenalisib, or IPI-549) described herein and/or CD47 inhibitor, formulated together with one or more pharmaceutically acceptable excipients. In some instances, the PI3K inhibitor (e.g., duvelisib, tenalisib, or IPI-549) or CD47 inhibitor disclosed herein are administered in separate pharmaceutical compositions and may (e.g., because of different physical and/or chemical characteristics) be administered by different routes (e.g., one therapeutic is administered orally, while the other is administered intravenously). In other instances, the PI3K inhibitor (e.g., duvelisib, tenalisib, or IPI-549) or CD47 inhibitor disclosed herein may be administered separately, but via the same route (e.g., both orally or both intravenously). In still other instances, the PI3K inhibitor (e.g., duvelisib, tenalisib, or IPI-549) or CD47 inhibitor disclosed herein may be administered in the same pharmaceutical composition.

The selected dosage level will depend upon a variety of factors including, for example, the activity of the particular compound employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of PI3K inhibitor (e.g., duvelisib, tenalisib, or IPI-549) described herein and/or a CD47 inhibitor will be that amount of the compound which, in some embodiments, may be the lowest dose effective to produce a therapeutic effect. Such an effective dose will generally depend upon the factors described herein. Generally, doses of a PI3K inhibitor (e.g., duvelisib, tenalisib, or IPI-549) or a CD47 inhibitor described herein for a patient, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to 1000 mg, or about 0.01 mg to about 500 mg per day, or about 0.1 mg to about 500 mg per day, or about 1 mg to 50 mg per day, or about 5 mg to 40 mg per day, An exemplary dosage is about 10 to 75 mg (e.g., 10 tug, 25 mg, 50 mg, 75 mg) per day. An exemplary dosage is about 10 to 75 mg (e.g., 10 mg, 25 mg, 50 mg, 75 mg) twice per day. An exemplary dosage is about 10 to 30 mg (e.g., 10 mg, 1.5 mg, 20 mg, 25 mg, 30 mg) per day. Another exemplary dosage is about 10 to 30 mg (e.g., 10 mg, 15 mg, 20 mg, 25 mg, 30 mg) twice per day. In some embodiments, for a. 70 kg human, a suitable dose would be about 0.05 to about 7 g/day, such as about 0.05 to about 2.5 g/day. Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, the compounds may be administered daily, every other day, three times a week, twice a week, weekly, or bi-weekly. The dosing schedule can include a “drug holiday,” e.g., the drug may be administered for two weeks on, one week off, or three weeks on, one week off, or four weeks on, one week off, etc., or continuously, without a drug holiday. The compounds may be administered orally, intravenously, intraperitoneally, topically, transdermally, intramuscularly, subcutaneously, intranasally, sublingually, or by any other route.

In some embodiments, PI3K inhibitor (e.g., duvelisib, tenalisib, or IPI-549) or the CD47 inhibitor described herein may be administered in multiple doses. Dosing may be about once, twice, three times, four times, five times, six times, or more than six tunes per day. Dosing may be about once a month, about once every two weeks, about once a week, or about once every other day. In some cases, continuous dosing is achieved and maintained as long as necessary.

Administration of the pharmaceutical compositions as disclosed herein may continue as long as necessary. In some embodiments, an agent as disclosed herein is administered for more than about 1, about 2, about 3, about 4, about 5, about 6, about 7, about 14, or about 28 days. In some embodiments, an agent as disclosed herein is administered for less than about 28, about 14, about 7, about 6, about 5, about 4, about 3, about 2, or about 1 day. In some embodiments, a therapeutic agent as disclosed herein is administered chronically on an ongoing basis, e.g., for the treatment of chronic effects.

Since a PI3K inhibitor (e.g., duvelisib, tenalisib, or IPI-549) may be administered in combination with a CD47 inhibitor, the doses of each agent or therapy may be lower than the corresponding dose for single-agent therapy. The dose for single-agent therapy can range from, for example, about 0.0001 to about 200 mg, or about 0.001 to about 100 mg, or about 0.01 to about 100 mg, or about 0.1 to about 100 mg, or about 1 to about 50 mg per kilogram of body weight per day.

### Kits

In some embodiments, provided herein are kits. The kits may include a pharmaceutical composition as described herein, in suitable packaging, and written material that can include instructions fair use, discussion of clinical studies, listing of side effects, and the like. Such kits may also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information may be based on the results of various studies, for example, studies using experimental animals involving in vivo models and studies based on human clinical trials.

In some embodiments, a memory aid is provided with the kit, e.g., in the form of numbers next to the tablets or capsules whereby the numbers correspond with the day's of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” may be a single tablet or capsule or several tablets or capsules to be taken on a given day.

The kit may contain a inhibitor (e.g., duvelisib, tenalisib, or IPI-549) and a CD47 inhibitor. In some embodiments, the PI3K inhibitor (e.g., duvelisib, tenalisib, or IPI-549) and the CD47 inhibitor are provided as separate pharmaceutical compositions in separate containers within the kit. In some embodiments, the PI3K inhibitor (e.g., duvelisib, tenalisib, or IPI-549) and the CD47 inhibitor are provided as a single pharmaceutical composition within a container in the kit. Suitable packaging and additional articles for use (e.g., measuring cup for liquid preparations, foil wrapping to minimize exposure to air, and the like) are known in the art and may be included in the kit. In other embodiments, kits may further comprise devices that are used to administer the active agents. Examples of such devices include, but are not limited to, syringes, drip bags, patches, and inhalers. Kits described herein may be provided, marketed and/or promoted to health providers, including physicians, nurses, pharmacists, formulary officials, and the like. Kits can also, in some embodiments, be marketed directly to the consumer.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules may be removed from the blister pack by manually applying pressure on the recesses whereby an opening is formed in the sheet at the place of the recess. The tablet or capsule can then be removed via said opening,

Kits may further comprise pharmaceutically acceptable vehicles that may be used to administer one or more active agents. For example, if an active agent is provided in a solid form that must be reconstituted for parenteral administration, the kit can comprise a sealed container of a suitable vehicle in which the active agent may be dissolved to form a particulate-free sterile solution that is suitable for parenteral administration.. Examples of pharmaceutically acceptable vehicles include, but are not limited to: Water for Injection USP; aqueous vehicles such as, but not limited to, Sodium Chloride injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The present disclosure further encompasses anhydrous pharmaceutical compositions and dosage forms comprising an active ingredient, since water can facilitate the degradation of some compounds. For example, water may be added (e.g., about 5%) in the pharmaceutical arts as a means of simulating long-term storage in order to determine characteristics such as shelf-life or the stability of formulations over time. Anhydrous pharmaceutical compositions and dosage forms may be prepared using anhydrous or low moisture containing ingredients and low moisture or low humidity conditions. For example, pharmaceutical compositions and dosage forms which contain lactose may be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous pharmaceutical compositions may be packaged using materials known to prevent exposure to water such that they may be included in suitable formulary kits. Examples of suitable packaging include, but are not limited to, hermetically sealed foils, plastic or the like, unit dose containers, blister packs, and strip packs.

### Definitions

Unless defined otherwise, all technical and scientific terms used herein have the same meaning as is commonly understood by one of skill in the art to which this specification pertains.

As used in the specification and claims, the singular form “a” “an” and “the” includes plural references unless the context clearly dictates otherwise.

As used herein, and unless otherwise indicated, the term “about” or “approximately” means an acceptable error for a particular value as determined by one of ordinary skill in the art, which depends in part on how the value is measured or determined. In some embodiments, the term “about” or “approximately” means within 1, 2, 3, or 4 standard deviations. In some embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “antagonist” and “inhibitor” are used interchangeably, and they refer to a compound or agent having the ability to reduce or inhibit a biological function of a target protein or polypeptide, such as by reducing or inhibiting the activity or expression of the target protein or polypeptide. Accordingly, the terms “antagonist” and “inhibitor” are defined in the context of the biological role of the target protein or polypeptide. An inhibitor need not completely abrogate the biological function of a target protein or polypeptide, and in some embodiments reduces the activity by at least 50%, 60%, 70%, 80%, 90%, 95%, or 99%. While some antagonists herein specifically interact with (e.g., bind to) the target, compounds that inhibit a biological activity of the target protein or polypeptide by interacting with other members of the signal transduction pathway of which the target protein or polypeptide are also specifically included within this definition. Non-limiting examples of biological activity inhibited by an antagonist include those associated with the development, growth, or spread of a tumor, or an undesired immune response as manifested in autoimmune disease.

The term “effective amount” or “therapeutically effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the terms “treatment” and “treating” are used herein to refer to an approach for obtaining beneficial or desired results including, but not limited to, therapeutic benefit. A therapeutic benefit includes, but is not limited to, eradication, inhibition, reduction, or amelioration of the underlying disorder being treated. Also, a therapeutic benefit is achieved with the eradication, inhibition, reduction, or amelioration of one or more of the physiological symptoms associated with the underlying disorder such that an improvement is observed in the patient, notwithstanding that the patient can still be afflicted with the underlying disorder.

As used herein, the terms “prevention” and “preventing” are used herein to refer to an approach for obtaining beneficial or desired results including, but not limited, to prophylactic benefit. For prophylactic benefit, the pharmaceutical compositions may be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action. The term “therapeutically effective agent” or “therapeutic agent” means a composition that will elicit the biological or medical response of a tissue, system, animal or human that is being sought by the researcher, veterinarian, medical doctor or other clinician.

“Subject” or “patient” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

The term “selective inhibition” or “selectively inhibit” as applied to a biologically active agent refers to the agent's ability to selectively reduce the target signaling activity as compared to off-target signaling activity, via direct or indirect interaction with the target. For example, a compound that selectively inhibits one isoform of PI3K over another isoform of PI3K has an activity of at least greater than about 1× against a first isoform relative to the compound's activity against the second isoform (e.g., at least about 2×, 3×, 5×, 10×, 20×, 50×, 100×, 200×, 500×, or 1000×). In certain embodiments, these terms refer to (1) a compound described herein that selectively inhibits the gamma isoform over the alpha, beta, or delta isoform; or (2) a compound described herein that selectively inhibits the delta isoform. over the alpha, beta, or gamma isoform. By way of non-limiting example, the ratio of selectivity can be greater than a factor of about 1, greater than a factor of about 2, greater than a factor of about 3, greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by IC₅₀. In certain embodiments, the IC50 can be measured by in vitro or in vivo assays.

The term “in vivo” refers to an event that takes place in a subject's body.

The term “in vitro” refers to an event that takes places outside of subject's body. For example, an in vitro assay encompasses any assay conducted outside of a subject In vitro assays encompass cell-based assays in which cells, alive or dead, are employed. In vitro assays also encompass a cell-free assay in which no intact cells are employed.

“Combination therapy”, or “in combination with” refer to the use of more than one compound or agent to treat a particular disorder or condition. For example, a PI3K inhibitor (e.g., duvelisib, tenalisib, or IPI-549) may be administered in combination with a CD47 inhibitor. By “in combination with,” it is not intended to imply that he other therapy and the PI3K inhibitor must be administered at the same time and/or formulated for delivery together, although these methods of delivery are within the scope of this disclosure. The PI3K inhibitor can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), the CD47 inhibitor. In general, each therapeutic agent will be administered at a dose and/or on a time schedule determined for that particular agent. The CD47 inhibitor can be administered with the PI3K inhibitor in a single composition or separately in a different composition.

The terms “co-administration of and “co-administering” and their grammatical equivalents, as used herein, encompass administration of two or more agents to subject so that both agents and/or their metabolites are present in the subject at the same or substantially the same time. In some embodiments, co-administration of a PI3K inhibitor with a CD47 inhibitor (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The CD47 inhibitor can be administered prior to, at the same time as, or subsequent to administration of the PI3K inhibitor, or in some combination thereof. Where the PI3K inhibitor is administered to the patient at repeated intervals, e.g., during a standard course of treatment, the additional agent can be administered prior to, at the same time as, or subsequent to, each administration of the PI3K inhibitor, or some combination thereof, or at different intervals in relation to the PI3K inhibitor treatment, or in a single dose prior to, at any time during, or subsequent to the course of treatment with the PI3K inhibitor. In some embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “monotherapy” refers to the use of an agent individually (also referred to herein as alone) (e.g., as a single compound or agent), e.g., without a second active ingredient to treat the same indication, e.g., cancer. For example, in this context, the term monotherapy includes the use of either the PI3K inhibitor or the CD47 inhibitor to treat the cancer.

The term “synergy” or “synergistic” encompasses a more than additive effect of a combination of two or more agents compared to their individual effects. In some embodiments, synergy or synergistic effect refers to an advantageous effect of using two or more agents in combination, e.g., in a pharmaceutical composition, or in a method of treatment. In some embodiments, one or more advantageous effects is achieved by using a PI3K inhibitor in combination with a CD47 inhibitor as described herein.

In some embodiments, the synergistic effect is that a lower dosage of one or both of the agents is needed to achieve an effect. For example, the combination can provide a selected effect, e.g., a therapeutic effect, when at least one of the agents is administered at a lower dosage than the dose of that agent that would be required to achieve the same therapeutic effect when the agent is administered as a monotherapy. In some embodiments, the combination of a PI3K inhibitor (e.g., duvelisib, tenalisib, or IM-549) and a CD47 inhibitor (as described herein) allows the PI3K inhibitor to be administered at a lower dosage than would be required to achieve the same therapeutic effect if the PI3K inhibitor were administered as a monotherapy. In some embodiments, the combination of a PI3K inhibitor (e.g., duvelisib, tenalisib, or IPI-549) and a CD47 inhibitor (as described herein) allows the CD47 inhibitor to be administered at a lower dosage than would be required to achieve the same therapeutic effect if the CD47 inhibitor were administered as a monotherapy.

In some embodiments, the synergistic effect is a reduction, prevention, delay, or decrease in the occurrence or the likelihood of occurrence of one or more side effects, toxicity, resistance, that would otherwise be associated with administration of a CD47 inhibitor.

In some embodiments, a synergistic effect refers to the combination of a PI3K inhibitor (e.g., duvelisib, tenalisib, or IPI-549), and a CD47 inhibitor (as described herein), results in a therapeutic effect greater than the additive effect of the PI3K inhibitor and the CD47 inhibitor.

In some embodiments, the synergistic effect is a reduction in resistance (e.g., a decrease in a measure of resistance or a decreased likelihood of developing resistance), or a delay in the development of resistance, to at least one of the agents (e.g., a CD47 inhibitor as described herein or a PI3K inhibitor as described herein).

IC₅₀ refers to a measure of the effectiveness of therapeutic agent in inhibiting cancer cells by 50%.

An “opsonizing agent” as used herein is any agent (e.g., “opsonizing antibody”) that can bind to a target cell (e.g., a cancer cell, a cell harboring an intracellular pathogen, etc) and opsonize the target cell. For example, any antibody that can bind to a target cell, where the antibody has an Fc region, is considered to be an agent that opsonizes a target cell. In some cases, the agent that opsonizes a target cell is an antibody, other than an anti-CD47 antibody, that binds to a target cell (e.g., an anti-tumor antibody, an anti-cancer antibody, an anti-infection antibody, and the like). In some embodiments, the combination as described herein (e.g., a PI3K inhibitor in combination with a CD47 inhibitor) is administered together with an opsonizing agent (e.g., opsonizing antibody). Opsonizing antibodies include, but are not limited to, an anti-CD20 antibody (e.g., rituximab, obinutuzumab, ofatumumab), an anti-EGFR antibody (e.g., cetuximab, panitumumab), an anti-HER2 antibody (e.g., trastuzumab, pertuzumab), an anti-CD38 antibody (e.g., daratumumab, isatuximab), an anti-CD19 antibody (e.g., tafasitamab), an anti-CD22 antibody (e.g., moxetumomab pasudotox), an anti-CCR4 antibody (e.g., mogamulizumab), or any other opsonizing antibody.

The term “anti-cancer effect” refers to the effect a therapeutic agent has on cancer, e.g., a decrease in growth, viability, or both of a cancer cell. The IC₅₀ of cancer cells can be used as a measure the anti-cancer effect.

The term “tumor” refers to any neoplastic cell growth and proliferation, whether malignant or benign, and any pre-cancerous and cancerous cells and tissues.

The term “solid tumor” as used herein, refers to an abnormal mass of tissue that usually does not contain cysts or liquid areas. Solid tumors may be benign or malignant. Different types of solid tumors are named for the type of cells that form them. Examples of solid tumors include, but are not limited to, sarcomas, carcinomas, and lymphomas.

The term “cancer” includes, but is not limited to, solid tumors and blood born tumors.

The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus.

Hematopoietic origin refers to involving cells generated during hematopoiesis, a process by which cellular elements of blood, such as lymphocytes, leukocytes, platelets, erythrocytes and natural killer cells are generated. Cancers of hematopoietic origin includes lymphoma and leukemia.

Resistant or refractive refers to when a cancer that has a reduced responsiveness to a treatment, e.g., up to the point where the cancer does not respond to treatment. The cancer can be resistant at the beginning of treatment, or it may become resistant during treatment. The cancer subject may have one or more mutations that cause it to become resistant to the treatment, or the subject may have developed such mutations during treatment. The term “refractory” can refer to a cancer for which treatment (e.g., chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

“Responsiveness,” to “respond” to treatment, and other forms of this term, as used herein, refer to the reaction of a subject to treatment with a therapeutic, e.g., a PI3K inhibitor or a CD47 inhibitor, alone or in combination, e.g., monotherapy or combination therapy. Responsiveness to a therapy, e.g., treatment with a PI3K inhibitor or a CD47 inhibitor, alone or in combination, can be evaluated by using any of the alterations/biomarkers disclosed herein and/or comparing a subject's response to the therapy using one or more clinical criteria, such as IWCLL 2008 (for CLL) described in, e.g., Hallek, M. et al. (2008) Blood 111(12): 5446-5456; RECIST criteria for solid tumors (Response Evaluation Criteria In Solid Tumors), and the like. Additional classifications of responsiveness are provided in Brown, J. R. (2014) Blood, 123(22): 3390-3397 and Chesson, B. D. et al. Journal of Clinical Oncology, 30(23): 2820-2822.

These criteria provide a set of published rules that define when cancer patients improve (“respond”), stay the same (“stable”) or worsen (“progression”) during treatments.

In another embodiment in solid tumors, a subject responds to treatment with a PI3K inhibitor in combination with a CD47 inhibitor if growth of a tumor in the subject is retarded about 10%, 20%, 30%, 40%, 50%, 60%, 70%, 80%, 90% or more. In another example, a subject responds to treatment with a PI3K inhibitor in combination with a CD47 inhibitor, if a tumor in the subject shrinks by about 5%, 10%, 20%, 30%, 40%, 50% or more as determined by any appropriate measure, e.g., by mass or volume. In another example, a subject responds to treatment with a PI3K inhibitor in combination with a CD47 inhibitor, if the subject experiences a life expectancy extended by about 5%, 10%, 20%, 30%, 40%, 50% or more beyond the life expectancy predicted if no treatment is administered. In another example, a subject responds to treatment with a PI3K inhibitor in combination with a CD47 inhibitor, if the subject has an increased disease-free survival, overall survival or increased bene to progression. Several methods can be used to determine if a patient responds to a treatment including the RECIST criteria, as set forth above.

### Chemical Definitions

As used herein, a “pharmaceutically acceptable form” of a disclosed compound includes, but is not limited to, pharmaceutically acceptable salts, hydrates, solvates, isomers, prodrugs, and isotopically labeled derivatives of disclosed compounds. In some embodiments, a “pharmaceutically acceptable form” includes, but is not limited to, pharmaceutically acceptable salts, isomers, prodrugs and isotopically labeled derivatives of disclosed compounds.

In some embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J Pharmaceutical Sciences (1977) 66: 1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfa ethane sulfonate, formate, fumarate, glucoheptonate, glycerophosphate, gluconate, hemisulfate, heptanoate, hexanoate, hydroiodide, 2-hydroxy-ethane sulfonate, lactobionate, lactate, laurate, lauryl sulfate, malate, maleate, malonate, methanesulfonate, 2-naphthalene sulfonate, nicotinate, nitrate, oleate, oxalate, palmitate, pamoate, pectinate, persulfate, 3-phenylpropionate, phosphate, picrate, pi valate, propionate, stearate, succinate, sulfate, tartrate, thiocyanate, p-toluene sulfonate, undecanoate, valerate salts, and the like. In some embodiments, organic acids from which salts may be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts may be derived include, for example, primary, secondary, and tertiary amines, substituted amines including naturally occurring substituted amines, cyclic amines, basic ion exchange resins, and the like, such as isopropyl amine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In some embodiments, the pharmaceutically acceptable form is a solvate (e.g., a hydrate). As used herein, the term “solvate” refers to compounds that further include a stoichiometric or non-stoichiometric amount of solvent bound by non-covalent intermolecular forces. The solvate may be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or one to about 2, about 3 or about 4, solvent or water molecules. It will be understood that the term “compound” as used herein encompasses the compound and solvates of the compound, as well as mixtures thereof.

In some embodiments, the pharmaceutically acceptable form is a prodrug. As used herein, the term “prodrug” refers to compounds that are transformed in vivo to yield a disclosed compound or a pharmaceutically acceptable form of the compound. A prodrug may be inactive when administered to a subject, but is converted in vivo to an active compound, for example, by hydrolysis (e.g., hydrolysis in blood). In certain cases, a prodrug has improved physical and/or delivery properties over the parent compound. Prodrugs are typically designed to enhance pharmaceutically and/or pharmacokinetically based properties associated with the parent compound. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al, “Pro-drugs as Novel Delivery Systems,” A.C.S. Symposium Series, Vol. 1.4, Chp 1, pp 1-12 and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it enhances absorption from the digestive tract, or it can enhance drug stability for long-term storage.

The term “prodrug” is also meant to include any covalently bonded carriers, which release the active compound in vivo when such prodrug is administered to a subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed., 1995), and Design of Prodrugs (H. Bundgard ed., Elsevier, New York, 1985).

For example, if a disclosed compound or a pharmaceutically acceptable form of the compound contains a carboxylic acid functional group, a prodrug can comprise a pharmaceutically acceptable ester formed by the replacement of the hydrogen atom of the acid group with a group such as (C₂-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidine-, pyrrolidino- or morpholino(C₂-C₃)alkyl.

Similarly, if a disclosed compound or a pharmaceutically acceptable form of the compound contains an alcohol functional group, a prodrug may be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or a-aminoacyl-α-aminoacyl, where each α-aminoacyl group is independently selected from naturally occurring L-amino acids, P(O)(OH)₂, —P(O)(O(C₁-C₆)alkyl)₂, and glycosyl (the radical resulting from the removal of a hydroxyl group of the hemiacetal form of a carbohydrate).

If a disclosed compound or a pharmaceutically acceptable form of the compound incorporates an amine functional group, a prodrug may be formed by the replacement of a hydrogen atom in the amine group with a group such as R-carbonyl, RO-carbonyl, NRR′-carbonyl where R and are each independently (C₁-C₁₀)alkyl, (C₃-C₅)cycloalkyl, benzyl, a natural α-aminoacyl or natural α-aminoacyl-natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N-or di-N,N-(C₁-C₆)alkylaminoalkyl, —C(Y⁴)Y⁵ wherein Y⁴ is H or methyl and Y⁵ is mono-N- or di-N,N-(C₁-C₆)alkylamino, morpholine, piperidin-1-yl or pyrrolidin-1-yl.

In some embodiments, the pharmaceutically acceptable form is an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown may be designated (+) or (−) depending on the direction (dextro-or levorotatory) which they rotate plane polarized light at the wavelength of the sodium D line. Certain of the compounds described herein contain one or more asymmetric centers and can thus give rise to enantiomers, diastereomers, and other stereoisomeric forms that may be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)- isomers may be prepared using chiral synthons or chiral reagents, or resolved using conventional techniques. When the compounds described herein contain (Actinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which can potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)- and 20% (R)-, the enantiomeric purity of the compound with respect to the (S)-isomeric form is 80%. The enantiomeric purity of a compound may be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

In some embodiments, the pharmaceutically acceptable form is a tautomer. As used herein, the term “tautomer” is a type of isomer that includes two or more interconvertible compounds resulting from at least one formal migration of a hydrogen atom and at least one change in valency (e.g., a single bond to a double bond, a triple bond to a double bond, or a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers may be reached. Tautomerizations (i.e., the reaction providing a tautomeric pair) may be catalyzed by acid or base, or can occur without the action or presence of an external agent. Exemplary tautomerizations include, but are not limited to, keto-enol; amide-imide; lactam-lactim; enamine-imine; and enamine-(a different) enamine tautomerizations. A specific example of keto-enol tautomerization is the interconversion of pentane-2,4-diene and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Unless otherwise stated, structures depicted herein are also meant to include compounds which differ only in the presence of one or more isotopically enriched atoms. For example, compounds having the present structures except for the replacement or enrichment of a hydrogen by deuterium or tritium at one or more atoms in the molecule, or the replacement or enrichment of a carbon by ¹³C or ¹⁴C at one or more atoms in the molecule, are within the scope of this disclosure. In some embodiments, provided herein are isotopically labeled compounds having one or more hydrogen atoms replaced by or enriched by deuterium. In some embodiments, provided herein are isotopically labeled compounds having one or more hydrogen atoms replaced by or enriched by tritium. In some embodiments, provided herein are isotopically labeled compounds having one or more carbon atoms replaced or enriched by ¹³C. In some embodiments, provided herein are isotopically labeled compounds having one or more carbon atoms replaced or enriched by ¹⁴C.

The disclosure also embraces isotopically labeled compounds which are identical to those recited herein, except that one or more atoms are replaced by an atom having an atomic mass or mass number different from the atomic mass or mass number usually found in nature. Examples of isotopes that may be incorporated into disclosed compounds include isotopes of hydrogen, carbon, nitrogen, oxygen, phosphorus, sulfur, fluorine, and chlorine, such as, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. Certain isotopically-labeled disclosed compounds (e.g., those labeled with ³H and/or ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure.

As used herein, and unless otherwise specified, “polymorph” may be used herein to describe a crystalline material, e.g., a crystalline form. In some embodiments, “polymorph” as used herein are also meant to include all crystalline and amorphous forms of a compound or a salt thereof, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms, as well as mixtures thereof, unless a particular crystalline or amorphous form is referred to. Compounds of the present disclosure include crystalline and amorphous forms of those compounds, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms of the compounds or a salt thereof, as well as mixtures thereof.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions as disclosed herein is contemplated. Supplementary active ingredients can also be incorporated into the pharmaceutical compositions.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### EXAMPLES

The representative examples that follow are intended to help illustrate the invention, and are not intended to, nor should they be construed to, limit the scope of the invention.

### Example 1

### Duvelisib in Combination with Anti-CD47 (Clone B6H12) and an Opsonizing Antibody

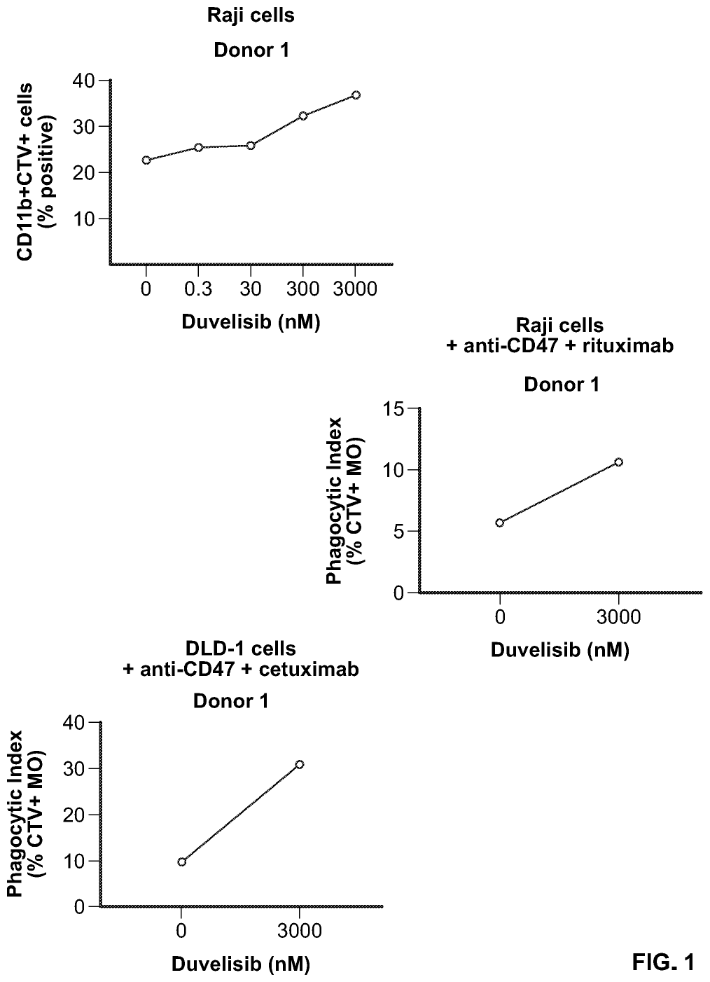

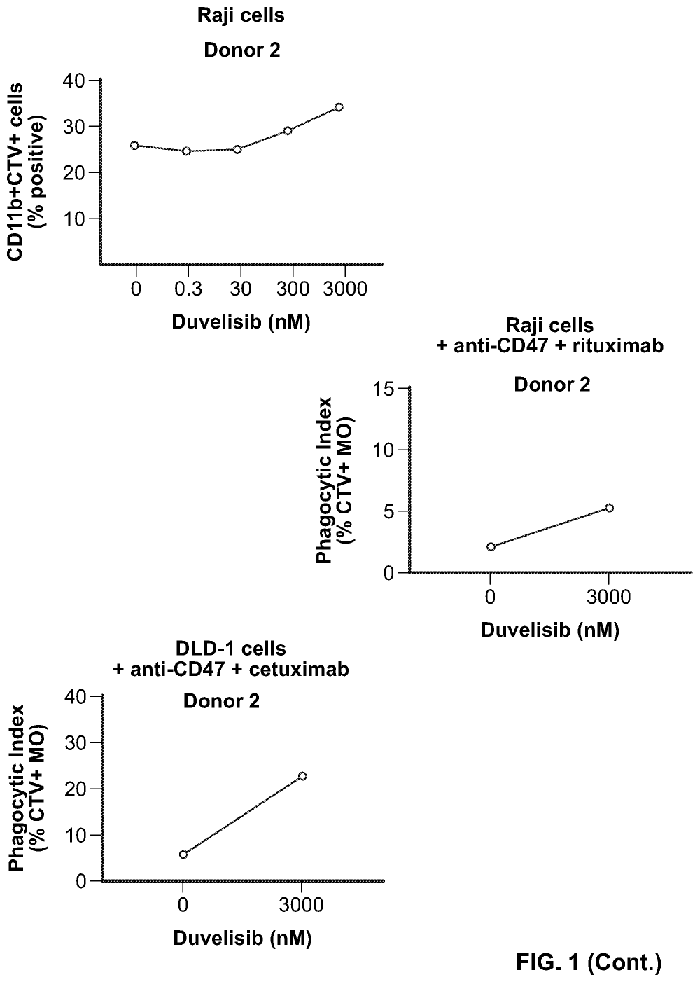

PBMC were isolated using a density gradient from healthy donor blood samples. CD14+ monocytes were then separated using magnetic selection and differentiated into MO macrophages for 6 days in the presence of M-CSF and 3000 nM of duvelisib or vehicle (DMSO). Two cell lines positive for surface expression of CD47, DLD-1 (ATCC® CCL-221™) and Raji cells (ATCC® CCL-86™), were labelled with cell trace violet (CTV). These were then offered to the macrophage cultures in the presence of duvelisib or vehicle control for 4 hours to allow phagocytosis to occur. For the part of the assay where a CD47 blocking antibody and an opsonizing antibody were used, CD47 blocking antibody at 1 μg/ml (clone B6H12) and rituximab (for Raji cells) or cetuximab (for DLD-1 cells) at 1 μg/ml were added during the 4 hours co-culture. The effect of duvelisib on the capacity of macrophages to phagocytose target cells was determined in a flow cytometry-based assay. FIG. 1 shows the results of the study in two representative donors per condition. As shown in FIG. 1, duvelisib increases phagocytosis by human macrophages as a single agent, and in combination with anti-CD47 (clone B6H12) and an opsonizing antibody (rituximab or cetuximab).

### Example 2

### Duvelisib in Combination with Anti-CD47 (Clone 5F9)

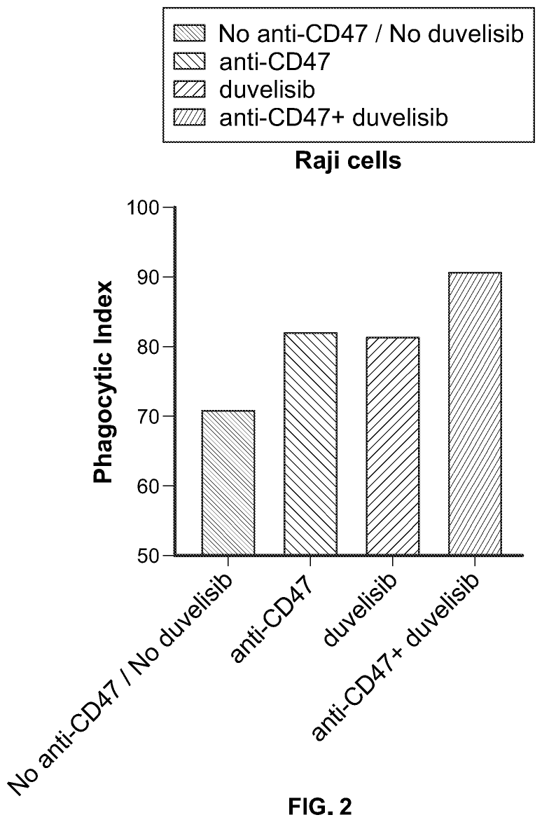

PBMC were isolated using a density gradient from healthy donor blood samples. CD14+ monocytes were then separated using magnetic selection and differentiated into MO macrophages for 6 days in the presence of M-CSF. Duvelisib (3000 nM) or vehicle (DMSO) were added during the last 3 days of differentiation. Raji cells (ATCC® CCL-86™), which are positive for surface expression of CD47, were labelled with cell trace violet (CTV). These cells were then offered to the macrophage cultures in the presence of CD47 blocking antibody at 300 ng/ml (clone 5F9) for 2 hours to allow phagocytosis to occur. The effect of duvelisib on the capacity of macrophages to phagocytose target cells was determined in a flow cytometry-based assay. FIG. 2 shows the results of the study. As shown in FIG. 2, duvelisib enhances phagocytosis of Raji Burkitt's lymphoma cells by human macrophages in combination with anti-CD47 (clone 549) compared to duvelisib or anti-CD47 alone.

These results show that duvelisib enhances phagocytosis in response to at least two different anti-CD47 antibodies (B6H12 as in FIG. 1 and Hu-5F9 as in FIG. 2) in the presence or absence of an opsonizing antibody.

1. A method of treating, managing, or preventing cancer in a subject comprising administering to the subject a therapeutically effective amount of a PI3K inhibitor in combination with a CD47 inhibitor.

2. The method of claim 1, wherein the CD47 inhibitor reduces CD47/SIRPα interaction.

3. The method of claim 1, wherein the CD47 inhibitor is a protein comprising a CD47 binding domain of SIRPα linked to an immunoglobulin Fc region.

4. The method of claim 1, wherein the CD47 inhibitor is an anti-CD47 antibody.

5. The method of any one of claims 1 to 4, wherein the CD47 inhibitor is chosen from B6H12, Hu5F9-G4, TTI-621, CC-90002, TI-061, ALX-148, SRF-231, IF8 Ab, or 13H3 Ab, or a combination thereof.

6. The method of claim 5, wherein the CD47 inhibitor is 13H3 Ab.

7. The method of any one of claims 1 to 6, wherein the PI3K inhibitor is a PI3K-gamma inhibitor, a PI3K-delta inhibitor, or a PI3K-delta/gamma dual inhibitor.

8. The method of claim 7, wherein the PI3K inhibitor is a PI3K-gamma inhibitor.

9. The method of any one of claims 1 to 7, wherein the PI3K inhibitor is chosen from tenalisib, duvelisib, idelalisib, copanlisib, IPI-549, CAL-130, BKM 120, GDC-0941, PX-866, GDC-0032, BAY 80-6946, BEZ235, BYL719, BGT-226, GDC-0980, GSK 2126458, PF-05212384, XL765, AS604850, AS252424, or XL147, or a combination thereof.

10. The method of claim 9, wherein the PI3K inhibitor is tenalisib.

11. The method of claim 9, wherein the PI3K inhibitor is IPI-549.

12. The method of claim 9, wherein the PI3K inhibitor is duvelisib.

13. The method of any one of claims 1 to 12, wherein the PI3K inhibitor and the CD47 inhibitor are the only therapeutically active ingredients.

14. The method of any one of claims 1 to 12, wherein the PI3K inhibitor and the CD47 inhibitor are in a single dosage form.

15. The method of any one of claims 1 to 12, wherein the PI3K inhibitor and the CD47 inhibitor are in separate dosage forms.

16. The method of any one of claims 1 to 15, wherein the PI3K inhibitor and the CD47 inhibitor is synergistic in treating a cancer.

17. The method of any one of claims 1 to 16, wherein the anti-cancer effect provided by a combination of the PI3K inhibitor and the CD47 inhibitor is greater than the anti-cancer effect relative to a monotherapy with the same dose of the PI3K inhibitor.

18. The method of any one of claims 1 to 16, wherein the anti-cancer effect provided by a combination of the PI3K inhibitor and the CD47 inhibitor is greater than the anti-cancer effect relative to a monotherapy with the same dose of the CD47 inhibitor.

19. The method of any one of claims 1 to 18, wherein the PI3K inhibitor is administered concurrently with the CD47 inhibitor.

20. The method of any one of claims 1 to 18, wherein the PI3K inhibitor is administered subsequent to the CD47 inhibitor.

21. The method of any one of claims 1 to 18, wherein the PI3K inhibitor is administered prior to the CD47 inhibitor.

22. A method of improving the efficacy of a CD47 inhibitor, the method comprising administering to a subject having cancer a therapeutically effective amount of a PI3K inhibitor in combination with the CD47 inhibitor.

23. A method of improving the efficacy of a CD47 inhibitor, the method comprising:
(a) administering to a subject having cancer a therapeutically effective amount of a CD47 inhibitor for a first period of time;
(b) after the first period of time, administering to the subject having cancer a therapeutically effective amount of a combination therapy comprising the PI3K inhibitor in combination with a CD47 inhibitor for a second period of time; and
(c) optionally repeating steps (a) and (b) one or more times.

24. A method of reducing a hematologic-related side effect and/or toxicity in a subject having cancer and receiving treatment with a CD47 inhibitor for the cancer, the method comprising administering to the subject a therapeutically effective amount of a PI3K inhibitor in combination with the CD47 inhibitor.

25. A method of delaying or decreasing resistance of a subject having cancer, the method comprising administering to the subject a therapeutically effective amount of a PI3K inhibitor in combination with a CD47 inhibitor, wherein the resistance is resistance to a reduction of CD47/SIRPα interaction.

26. The method of any one of claims 22 to 25, wherein the CD47 inhibitor reduces CD47/SIRPα interaction.

27. The method of any one of claims 22 to 25, wherein the CD47 inhibitor is a protein comprising a CD47 binding domain of SIRPα linked to an immunoglobulin Fc region.

28. The method of any one of claims 22 to 25, wherein the CD47 inhibitor is an anti-CD47 antibody.

29. The method of any one of claims 22 to 25, wherein the CD47 inhibitor is chosen from B6H12, Hu5F9-G4, TTI-621, CC-90002, TI-061, ALX-148, SRF-231, IF8 Ab, or 13H3 Ab, or a combination thereof.

30. The method of claim 29, wherein the CD47 inhibitor is 13H3 Ab.

31. The method of any one of claims 22 to 30, wherein the PI3K inhibitor is a PI3K-gamma inhibitor, PI3K-delta inhibitor, or PI3K-delta/gamma dual inhibitor.

32. The method of claim 31, wherein the PI3K inhibitor is a PI3K-gamma inhibitor.

33. The method of any one of claims 22 to 30, wherein the PI3K inhibitor is chosen from tenalisib, duvelisib, idelalisib, copanlisib, IPI-549, CAL-130, BKM 120, GDC-0941 866, GDC-0032, BAY 80-6946, BEZ235, BYL719, BGT-226, GDC-0980, GSK 2126458, PF-05212384 XL765, AS604850, AS252424, or XL147 or a combination thereof.

34. The method of claim 33, wherein the PI3K inhibitor is tenalisib.

35. The method of claim 33, wherein the PI3K inhibitor is IPI-549.

36. The method of claim 33, wherein the PI3K inhibitor is duvelisib.

37. The method of any one of claims 1 to 36, wherein the cancer is of hematopoietic origin.

38. The method of claim 37, wherein the cancer is lymphoma or leukemia.

39. The method of claim 37, wherein the cancer is B-cell lymphoma, mantle cell lymphoma, non-Hodgkin's lymphoma, non-Hodgkin's B-cell lymphoma, or T-cell lymphoma.

40. The method of claim 37, wherein the cancer is multiple myeloma.

41. The method of claim 37, wherein the cancer is non-Hodgkin's lymphoma.

42. The method of claim 41, wherein the non-Hodgkin's lymphoma is B-cell non-Hodgkin's lymphoma.

43. The method of claim 42, wherein the B-cell non-Hodgkin's lymphoma is diffuse large B-cell lymphoma.

44. The method of claim 43, wherein the diffuse large B-cell lymphoma is diffuse large B-cell lymphoma activated B-cell-like or diffuse large B-cell lymphoma germinal center B-cell-like.

45. The method of claim 42, wherein the B-cell non-Hodgkin's lymphoma is marginal zone lymphoma.

46. The method of claim 37, wherein the cancer is indolent non-Hodgkin's lymphoma.

47. The method of claim 46, wherein the indolent non-Hodgkin's lymphoma is chronic lymphocytic leukemia/small lymphocytic lymphoma.

48. The method of claim 37, wherein the cancer is follicular lymphoma.

49. The method of claim 37, wherein the cancer is acute myeloid leukemia.

50. The method of claim 37, wherein the cancer is T-cell lymphoma.

51. The method of claim 50, wherein the T-cell lymphoma is peripheral T-cell lymphoma.

52. The method of any one of claims 1 to 36, wherein the cancer is a solid tumor.

53. The method of claim 52, wherein the solid tumor is an ovarian cancer, pancreatic cancer, breast cancer, prostate cancer, colorectal cancer, non-small cell lung cancer, squamous cell carcinoma, or hepatocellular carcinoma.

54. The method of any one of claims 1 to 53, wherein the subject is a human.

55. The method of any one of claims 1 to 54, further comprising administering an opsonizing antibody to the subject.

56. The method of claim 55, wherein the opsonizing antibody is an anti-CD20 antibody (e.g., rituximab, obinutuzumab, ofatumumab), an anti-EGFR antibody (e.g., cetuximab, panitumumab), an anti-HER2 antibody (e.g., trastuzumab, pertuzumab), an anti-CD38 antibody (e.g., daratumumab, isatuximab), an anti-CD19 antibody (e.g., tafasitamab), an anti-CD22 antibody (e.g., moxetumomab pasudotox), or an anti-CCR4 antibody (e.g., mogamulizumab), or a combination thereof.

57. A composition comprising a combination of a PI3K inhibitor and a CD47 inhibitor.

58. The composition of claim 57, further comprising an opsonizing antibody.

59. The composition of claim 58, wherein the opsonizing antibody is an anti-CD20 antibody (e.g., rituximab, obinutuzumab, ofatumumab), an anti-EGFR antibody (e.g., cetuximab, panitumumab), an anti-HER2 antibody (e.g., trastuzumab, pertuzumab), an anti-CD38 antibody (e.g., daratumumab, isatuximab), an anti-CD19 antibody (e.g., tafasitamab), an anti-CD22 antibody (e.g., moxetumomab pasudotox), or an anti-CCR4 antibody (e.g., mogamulizumab), or a combination thereof.

60. The composition of any one of claims 57 to 59, wherein the CD47 inhibitor is an anti-CD47 antibody.

61. The composition of any one of claims 57 to 60, wherein the CD47 inhibitor is chosen from B6H12, Hu5F9-G4, TTI-621, CC-90002, TI-061, ALX-148, SRF-231, IF8 Ab, or 13H3 Ab, or a combination thereof.

62. The composition of claim 61, wherein the CD47 inhibitor is 13H3 Ab.

63. The composition of any one of claims 57 to 62, wherein the PI3K inhibitor is a PI3K-gamma inhibitor, PI3K-delta inhibitor, or PI3K-delta/gamma dual inhibitor.

64. The composition of claim 63, wherein the PI3K inhibitor is a PI3K-gamma inhibitor.

65. The composition of any one of claims 57 to 63, wherein the PI3K inhibitor is chosen from tenalisib, duvelisib, idelalisib, copanlisib, IPI-549, CAL-130, BKM 120, GDC-0941, PX-866, GDC-0032, BAY 80-6946, BEZ235, BYL719, BGT-226, GDC-0980, GSK 2126458, PF-05212384, XL765, AS604850, AS252424, or XL147, or a combination thereof.

66. The composition of claim 65, wherein the PI3K inhibitor is tenalisib.

67. The composition of claim 65, wherein the PI3K inhibitor is IPI-549.

68. The composition of claim 65, wherein the PI3K inhibitor is duvelisib.
